(19) United States
(12) Patent Application Publication
(10) Pub. No.: US 2025/0206713 A1
(43) Pub. Date: Jun. 26, 2025

(54) 2-[(2-OXO-4-PHENYL-2H-CHROMEN-7-YL)OXY] PROPANAMIDO DERIVATIVES

(71) Applicant: LUNELLA BIOTECH, INC., Ottawa, Ontario (CA)

(72) Inventors: Michael P. LISANTI, Didsbury Village, Greater Manchester (GB); Federica SOTGIA, Didsbury Village, Greater Manchester (GB); Jussi KANGASMETSA, Cambridge (GB); Filippo DI PISA, Sanford (GB)

(21) Appl. No.: 18/850,910

(22) PCT Filed: Mar. 23, 2023
(86) PCT No.: PCT/IB2023/052886

§ 371 Date: Sep. 25, 2024

Related U.S. Application Data
(60) Provisional application No. 63/325,360, filed on Mar. 30, 2022.

(51) Int. Cl. C07D 311/18; A61K 31/37; A61K 31/4025; A61K 31/453; A61P 35/04; C07D 405/12

(52) U.S. Cl. CPC C07D 311/18; A61K 31/37; A61K 31/4025; A61K 31/453; A61P 35/04; C07D 405/12

(57) ABSTRACT

Disclosed are 2-[(2-Oxo-4-phenyl-2H-chromen-7-yl)oxy]propanamido derivatives, wherein at least one of R1 to R4 bears an amido group. These derivatives may be used to target and eliminate cancer stem cells, and may be used as therapeutic agents for the treatment of cancer, including reducing the likelihood of and/or preventing tumor recurrence and metastasis. The compounds disclosed herein have demonstrated inhibition of tumorsphere forming capacity, migration and sternness-related signaling in cancer stem cells. These properties result from selective inhibiting of mitochondrial transcription targeting mitochondrial RNA polymerase (POLRMT) in cancer cells. Formula (II)

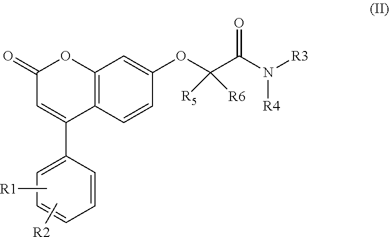

### FIELD

The present disclosure relates to 2-[(2-oxo-4-phenyl-2H-chromen-7-yl)oxy]propanamido derivatives that inhibit the propagation of cancer stem cells (CSCs) and senescent cells.

### BACKGROUND

The biological process of aging continues to receive significant attention in the scientific and medical research communities. Physiologic aging relates, at least in part, to an increase in the rate of oxidative damage to cellular components, including DNA, lipids, proteins, and the like. The increased oxidative damage creates an imbalance that disrupts self-regulating processes at the cellular level. Further, aging correlates to an accumulation of lipofuscin in neuron cytoplasm. Modern research also indicates that aging is a consequence of naturally occurring DNA damage, resulting in abnormal DNA alterations, accumulating over time. Both mitochondrial and nuclear DNA damage can contribute to aging, indirectly through increasing apoptosis and cellular senescence, and directly by increasing cell dysfunction. Accumulated DNA damage can lead to loss of cells and, in surviving cells, loss of gene expression and mutation—effects that, in infrequently dividing cells, produce indicia of aging. Cellular senescence results when aged cells cease cellular division, believed to occur following various environmental damaging events, abnormal cell growth, autophagy, and oxidative stress, among other factors. Senescence Associated Secretory Phenotype (“SASP”) is a characteristic of senescent cells, and lead to a proteotoxic impairment of healthy cell function, including inflammatory or anti-inflammatory and tumor or anti-tumor effects, depending on a host of factors. The impact of SASP-related chronic inflammation impacts the immune system's normal ability to remove senescent cells, and cells providing an immune function can be conscripted by SASP into senescent cells. Biomarkers of cellular senescence have been found to accumulate as mammals age, and contribute to a wide range of age-related diseases, including Alzheimer's, lateral sclerosis, and type 2 diabetes. And with respect to frequently dividing cells, accumulated DNA damage can become a prominent cause of cancer.

Aging thus increases the likelihood of developing cancer, and researchers have struggled to develop new anti-cancer and anti-aging or senolytic treatments. Conventional cancer therapies (e.g. irradiation, alkylating agents such as cyclophosphamide, and anti-metabolites such as 5-Fluorouracil) have attempted to selectively detect and eradicate fast-growing cancer cells by interfering with cellular mechanisms involved in cell growth and DNA replication. Other cancer therapies have used immunotherapies that selectively bind mutant tumor antigens on fast-growing cancer cells (e.g., monoclonal antibodies). Unfortunately, tumors often recur following these therapies at the same or different site(s), indicating that not all cancer cells have been eradicated. Cancer stem cells, in particular, survive for various reasons, and lead to treatment failure. Relapse may be due to insufficient chemotherapeutic dosage and/or emergence of cancer clones resistant to therapy. Hence, novel cancer treatment strategies are needed that overcome the deficiencies of conventional therapies.

Advances in mutational analysis have allowed in-depth study of the genetic mutations that occur during cancer development. Despite having knowledge of the genomic landscape, modern oncology has had difficulty with identifying primary driver mutations across cancer subtypes. The harsh reality appears to be that each patient's tumor is unique, and a single tumor may contain multiple divergent clone cells. What is needed, then, is a new approach that emphasizes commonalities between different cancer types. Targeting the metabolic differences between tumor and normal cells holds promise as a novel cancer treatment strategy. An analysis of transcriptional profiling data from human breast cancer samples revealed more than 95 elevated mRNA transcripts associated with mitochondrial biogenesis and/or mitochondrial translation. Sotgia et al., Cell Cycle, 11(23):4390-4401 (2012). Additionally, more than 35 of the 95 upregulated mRNAs encode mitochondrial ribosomal proteins (MRPs). Proteomic analysis of human breast cancer stem cells likewise revealed the significant overexpression of several mitoribosomal proteins as well as other proteins associated with mitochondrial biogenesis. Lamb et al., Oncotarget, 5(22):11029-11037 (2014).

Cancer cell mitochondrial metabolism has been the target of recent explorative research, with respect to both searching for anti-cancer therapeutic targets and senolytic therapeutic targets. Mitochondria are extremely dynamic organelles in constant division, elongation and connection to each other to form tubular networks or fragmented granules in order to satisfy the requirements of the cell and adapt to the cellular microenvironment. The balance of mitochondrial fusion and fission dictates the morphology, abundance, function and spatial distribution of mitochondria, therefore influencing a plethora of mitochondrial-dependent vital biological processes such as adenosine triphosphate (ATP) production, mitophagy, apoptosis, and calcium homeostasis. In turn, mitochondrial dynamics can be regulated by mitochondrial metabolism, respiration and oxidative stress.

ATP is the universal bioenergetic “currency” of all living cells and tissues, including microorganisms, such as prokaryotic bacteria and eukaryotic yeast. In eukaryotes, mitochondrial organelles function as the “powerhouse” of the cell. Mitochondria generate the vast amount of ATP via the TCA cycle and oxidative phosphorylation (OXPHOS), while glycolysis contributes a minor amount of ATP. Conversely, mitochondrial dysfunction induces ATP-depletion, resulting in mitochondrial-driven apoptosis (programmed cell death) and/or necrosis. Thus, we have proposed that ATP-depletion therapy may be a viable strategy for targeting and eradicating even the “fittest” cancer cells.

In MCF7 breast cancer cells, mitochondrial-driven OXPHOS contributes to 80-90% of ATP production, while glycolysis only contributes the remaining 10-20%, under normoxic conditions. Therefore, like normal cells, cancer cells are highly dependent on mitochondrial ATP production. However, it still remains largely unknown if ATP levels in cancer cells contribute to undergo 3D anchorage-independent growth and cell migration, two characteristic features of metastatic spread.

What is needed, then, are novel anti-aging compositions and methods that treat aging at the cellular level, overcoming accumulated oxidative and DNA damage and the numerous undesired effects of aging.

Further, what is needed are therapeutic agents that target unhealthy senescent cells and SASP, reducing the accumulation of cellular senescence and offsetting chronic senescence.

What is further needed are therapeutic agents that target a broad range of CSCs, through a feature common to CSCs regardless of cancer type.

What is further needed are therapeutic agents that inhibit the propagation of CSCs, including the circulating tumor cells and tumor-initiating cells that have the potential to cause tumor recurrence and/or metastasis.

### SUMMARY

In view of the foregoing background, it is an object of this disclosure to describe therapeutic agents, or compounds, that may be used to reduce the accumulation of cellular senescence, and inhibit CSC propagation. It is an object of this disclosure to describe therapeutic agents for use in eradicating CSCs and senescent cells. It is an object of this disclosure to describe therapeutic agents for use in preventing and reducing the likelihood of tumor recurrence and metastasis. It is further an object of this disclosure to describe compositions, such as pharmaceutical compositions, and methods for treating and preventing cancer, including tumor recurrence and/or metastasis. It is also an object of this disclosure to describe compositions, such as pharmaceutical compositions, and methods for senolytic therapeutic agents.

Described herein are compounds that may be used as therapeutic agents having anti-cancer activity, pharmaceutical compositions containing such therapeutic agents, methods for synthesizing such compounds, and methods for treating cancer. Embodiments of the therapeutic agents described herein may be compared to the formula shown below, to demonstrate the unexpectedly advantageous benefits of the present approach:

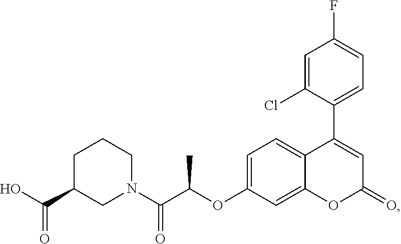 [SMILES: C[C@@H](Oc1ccc2c(-c3ccc(F)cc3Cl)cc(=O)oc2c1)C(=O)N1CCC[C@H](C(=O)O)C1]

a compound known to be an orally active, mitochondrial RNA polymerase (POLRMT) inhibitor. The compound was first described in Bonekamp, N. A., Peter, B., Hillen, H. S. et al. Small-molecule inhibitors of human mitochondrial DNA transcription. Nature 588, 712-716 (2020), herein incorporated by reference in its entirety. As a POLRMT inhibitor, the compound inhibits mitochondrial DNA expression. The IUPAC name for this embodiment is 3-piperidinecarboxylic acid, 1-[(2R)-2-[[4-(2-chloro-4-fluorophenyl)-2-oxo-2H-1-benzopyran-7-yl]oxy]-1-oxopropyl]-, (3S)-, and has been assigned CAS Registry No. 2304621-06-3.

Under the present approach, some embodiments of the compound may have the generic chemical structure of Formula 1, shown below:

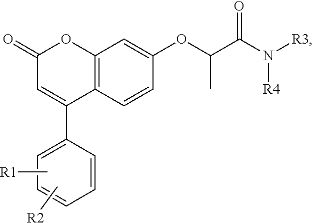

or pharmaceutically acceptable salts thereof, in which:
R1 and R2 may be the same or different, and are selected from a halogen, CF₂H, —CF₃, —OCF₂H, —OCF₃, substituted or unsubstituted C5-C18 carboxyl, substituted or unsubstituted C5-C18 alkane, substituted or unsubstituted C5-C18 alkene, substituted or unsubstituted C5-C18 cyclic alkene, substituted or unsubstituted C5-C18 alkyne, substituted or unsubstituted C5-C18 ketone, substituted or unsubstituted C5-C18 aldehyde, substituted or unsubstituted C5-C18 ether, substituted or unsubstituted C5-C18 ester, substituted or unsubstituted C5-C18 amine, substituted or unsubstituted C5-C18 amide, substituted or unsubstituted C5-C18 alkyl-amide, monocyclic or polycyclic arene, heteroarene, phenol, or benzoic acid; and
R3 and R4 may be the same or different, and are selected from hydrogen, substituted or unsubstituted C2-C18-alkyl, substituted or unsubstituted C3-C8-cycloalkyl, substituted or unsubstituted pyridine, substituted or unsubstituted C2-C18 carboxyl, substituted or unsubstituted C2-C18 alkene, substituted or unsubstituted C2-C18 alkyne, substituted or unsubstituted C2-C18 ketone, substituted or unsubstituted C2-C18 aldehyde, substituted or unsubstituted C2-C18 ether, substituted or unsubstituted C2-C18 ester, substituted or unsubstituted C2-C18 amine, substituted or unsubstituted C2-C18 amide, substituted or unsubstituted C2-C18 alkyl-amide, substituted or unsubstituted phenol, or benzoic acid, or one of R3 and R4 is the group

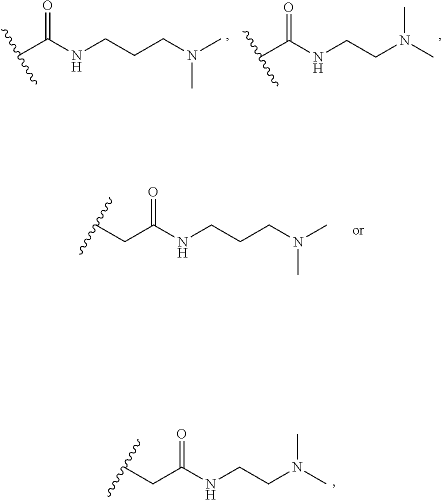

and the other is a H or a C2-C5 alkyl; or R3 and R4 form a substituted or unsubstituted C5 or C6 heterocycle; or R3 and R4 form the group

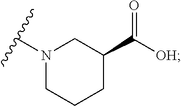 [SMILES: *N1CCC[C@H](C(=O)O)C1]

or NR3R4 is substituted with OH;
provided that at least one of R1 and R2 is a substituted or unsubstituted C5-C18 amide, or at least one of R3 and R4 is the group

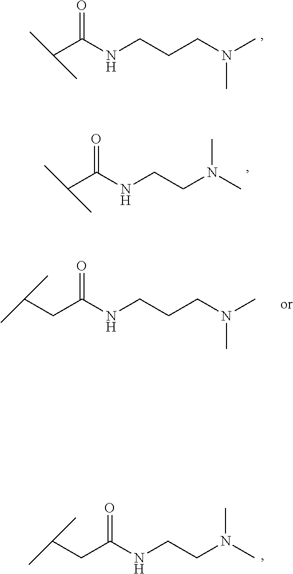

and the other is hydrogen or a C2-C5 alkyl.

It should be appreciated that the lactic acid moiety of the propanamido portion of Formula 1 may be modified with certain side chains—or the methyl replaced with hydrogen—in some embodiments. For example, some embodiments of compounds under the present approach have the generic chemical structure of Formula 2, shown below:

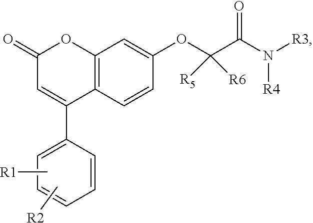

or pharmaceutically acceptable salts thereof, in which R1, R2, R3, and R4 are as described above with respect to Formula 1, and R5 and R6 may be the same or different, and are selected from hydrogen, substituted or unsubstituted C1-C10 alkyl, substituted or unsubstituted C3-C8-cycloalkyl, substituted or unsubstituted pyridine, substituted or unsubstituted C2-C10 carboxyl, substituted or unsubstituted C2-C10 alkene, substituted or unsubstituted C2-C10 alkyne, substituted or unsubstituted C2-C10 ketone, substituted or unsubstituted C2-C10 aldehyde, substituted or unsubstituted C2-C10 ether, substituted or unsubstituted C2-C10 ester, substituted or unsubstituted C2-C10 amine, substituted or unsubstituted C2-C10 amide, substituted or unsubstituted C2-C10 alkyl-amide, substituted or unsubstituted phenol, or benzoic acid.

The following paragraphs illustrate various examples of the present approach. It should be appreciated that the amide chain length may vary from C5 to C18. In some embodiments, for example, the therapeutic agent has the formula:

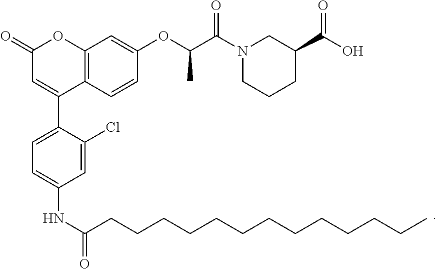 [SMILES: CCCCCCCCCCCCCC(=O)Nc1ccc(-c2cc(=O)oc3cc(O[C@H](C)C(=O)N4CCC[C@H](C(=O)O)C4)ccc23)c(Cl)c1]

As another example in some embodiments, the therapeutic agent has the formula:

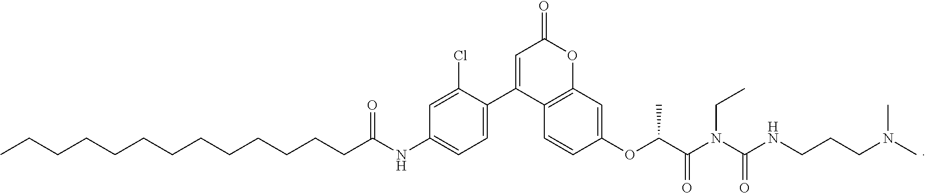 [SMILES: CCCCCCCCCCCCCC(=O)Nc1ccc(-c2cc(=O)oc3cc(O[C@H](C)C(=O)N(CC)C(=O)NCCCN(C)C)ccc23)c(Cl)c1]

As another example, some embodiments of the therapeutic agent have the formula:

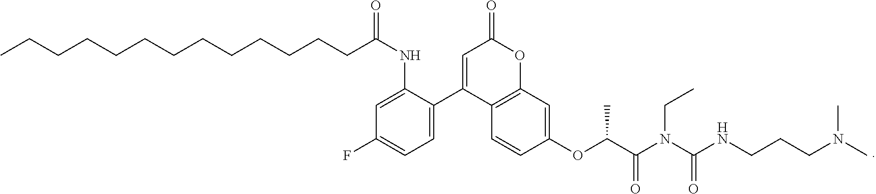 [SMILES: CCCCCCCCCCCCCC(=O)Nc1cc(F)ccc1-c1cc(=O)oc2cc(O[C@H](C)C(=O)N(CC)C(=O)NCCCN(C)C)ccc12]

As another example, some embodiments of the therapeutic agent have the formula:

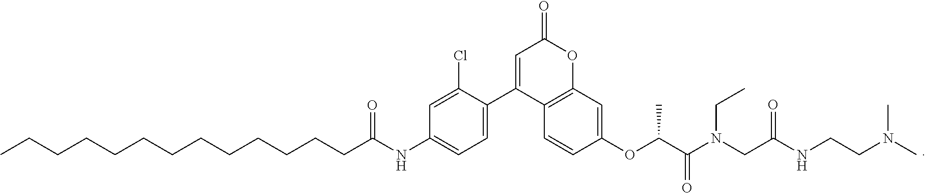 [SMILES: CCCCCCCCCCCCCC(=O)Nc1ccc(-c2cc(=O)oc3cc(O[C@H](C)C(=O)N(CC)CC(=O)NCCN(C)C)ccc23)c(Cl)c1]

In a further example, in some embodiments, the therapeutic agent has the formula:

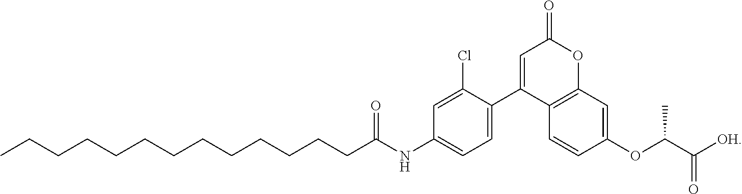 [SMILES: CCCCCCCCCCCCCC(=O)Nc1ccc(-c2cc(=O)oc3cc(O[C@H](C)C(=O)O)ccc23)c(Cl)c1]

As another example, some embodiments of the therapeutic agent have the formula:

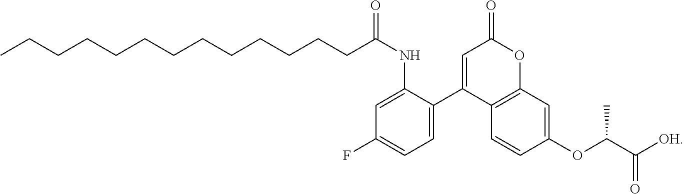 [SMILES: CCCCCCCCCCCCCC(=O)Nc1cc(F)ccc1-c1cc(=O)oc2cc(O[C@H](C)C(=O)O)ccc12]

As stated above, it should be appreciated that some embodiments of the therapeutic agent may have a C5-C18 amide. In laboratory testing, amides shorter than C5 and longer than C18 have limited therapeutic activity. In preferred embodiments, C10-C16 amides, and in particular C12-C15 amides, show considerably improved therapeutic activity relative to the baseline compound identified in paragraph [0014], above.

In some embodiments, one of R3 and R4 is the group

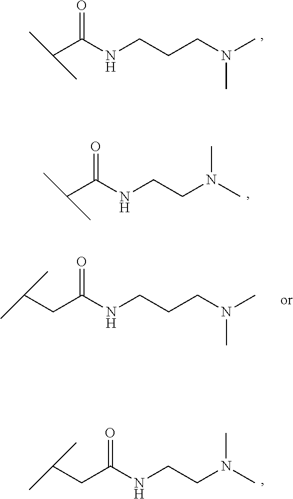

and the other is a H or a C2-C5 alkyl. For example, in the demonstrative structure below, R1 is Cl, R2 is iso-butylcarbamate, R3 is,

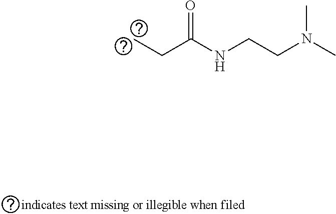

### R4 is C2.

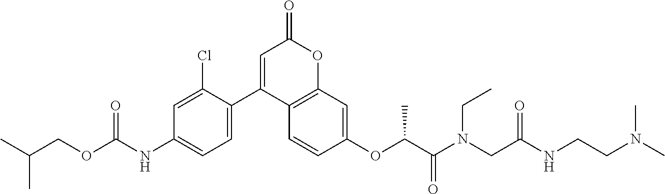 [SMILES: CCN(CC(=O)NCCN(C)C)C(=O)[C@@H](C)Oc1ccc2c(-c3ccc(NC(=O)OCC(C)C)cc3Cl)cc(=O)oc2c1]

In the example above, R2 may be varied as set forth above. For example, in another demonstrative structure, R2 may be tert-butylcarbamate.

The present approach may also be used to treat and/or prevent tumor recurrence and/or metastasis. Anti-cancer treatments often fail because the tumor recurs or metastasizes, particularly after surgery. CSC mitochondrial activity is understood to be, at least in part, responsible for these causes of treatment failure. Embodiments of the present approach may be used in situations where conventional cancer therapies fail, and/or in conjunction with or prior to anti-cancer treatments, to prevent or reduce the likelihood of treatment failure due to tumor recurrence and/or metastasis.

### BRIEF DESCRIPTION OF THE DRAWINGS

FIGS. 1A-1C show mammosphere formation results, SRB assay results on MCF-7 cells, and SRB assay results on BJ1-hTERT cells, for the Compound [I] embodiment.

FIGS. 2A-2C show mammosphere formation results, SRB assay results on MCF-7 cells, and SRB assay results on BJ1-hTERT cells, for the Compound [II] embodiment.

FIGS. 3A-3C show mammosphere formation results, SRB assay results on MCF-7 cells, and SRB assay results on BJ1-hTERT cells, for the Compound [III] embodiment.

FIGS. 4A and 4B show mammosphere formation results and SRB assay results on MCF-7 cells (2D), for the Compound [IV] embodiment.

FIGS. 5A-5C show mammosphere formation results, SRB assay results on MCF-7 cells, and SRB assay results on BJ1-hTERT cells, for the Compound [V] embodiment.

FIGS. 6A and 6B show mammosphere formation results and SRB assay results on MCF-7 cells, for the Compound [VI] embodiment.

FIGS. 7A and 7B show mammosphere formation results and SRB assay results on MCF-7 cells, for the Compound [VII] embodiment.

FIG. 8 shows a quantitative evaluation of tumor growth using the CAM assay, for different concentrations of an embodiment of the present approach.

FIG. 9 shows a quantitative evaluation of metastatic invasion using the CAM assay, for different concentrations of an embodiment of the present approach.

FIG. 10 shows a toxicity assessment using the CAM assay, for different concentrations of an embodiment of the present approach.

FIGS. 11A-D show mitochondrial respiration, basal respiration, maximal respiration, and ATP production results for metabolic flux analysis of adherent MCF7 cells were treated with a compound according to the present approach.

FIGS. 12A-D show the results of glycolytic function analysis for treatment with a compound according to the present approach.

### DESCRIPTION

The following description illustrates embodiments of the present approach in sufficient detail to enable practice of the present approach. Although the present approach is described with reference to these specific embodiments, it should be appreciated that the present approach can be embodied in different forms, and this description should not be construed as limiting any appended claims to the specific embodiments set forth herein. Rather, these embodiments are provided so that this disclosure will be thorough and complete, and will fully convey the scope of the present approach to those skilled in the art.

This description uses various terms that should be understood by those of an ordinary level of skill in the art. The following clarifications are made for the avoidance of doubt.

The term “cancer” refers to physiological conditions in mammals that are typically characterized by uncontrolled cell growth. This definition includes benign and malignant cancers. Examples of cancers include cancer types, lymphomas, blastomas (including medullablastomas and retinoblastomas), sarcomas (including liposarcomas and synovial sarcomas), neuroendocrine tumors (carcinoid tumors, gastrin production Includes, but is not limited to, tumors and islet cell carcinomas), sarcomas, Schwannomas (including acoustic neuroma), medullary carcinomas, adenocarcinomas, melanomas, and leukemia or lymphocyte tumors. Specific examples of cancers include bladder cancer, squamous cell carcinoma, small cell lung cancer, non-small cell lung cancer, lung adenocarcinoma, and lung cancer including squamous epithelial cancer of the lung, peritoneal cancer, hepatocellular carcinoma, gastric cancer including gastrointestinal cancer or stomach cancer, pancreatic cancer, glioblastoma, cervical cancer, ovarian cancer, liver cancer, bladder cancer, liver cancer, breast cancer (including metastatic breast cancer), colon cancer, rectal cancer, colon rectal cancer, endometrial cancer or uterine cancer, salivary adenocarcinoma, kidney cancer (kidney cancer) or kidney cancer (renal cancer), prostatic cancer, genital cancer, thyroid cancer, liver cancer, anal cancer, penis cancer, testicular cancer, esophageal cancer, bile duct tumor, and head and neck cancer and multiple myeloma.

As used herein, the term “tumor” refers to the growth and proliferation of neoplastic cells, whether malignant or benign, including pre-cancerous and cancerous cells and tissues.

The term “metastasis” refers to the spread of cancer from its primary site to other parts of the body. Cancer cells can escape from the primary tumor, penetrate lymph vessels and blood vessels, circulate through the bloodstream, and grow or “metastasize” in distant lesions in normal tissue elsewhere in the body. Metastases can be local or distant. Metastasis is a sequential process that requires tumor cells to escape from the primary tumor, travel through the bloodstream, and stop at distant sites. At this new site, cells can establish a blood supply and grow to form a life-threatening mass. Both irritating and inhibitory molecular pathways within tumor cells control this behavior, and the interaction between tumor cells and host cells at distant sites is also important.

The terms “treat,” “treated,” “treating,” and “treatment” include the diminishment or alleviation of at least one symptom associated or caused by the state, disorder or disease being treated, in particular, cancer. In certain embodiments, the treatment comprises diminishing and/or alleviating at least one symptom associated with or caused by the cancer being treated, by the compound of the invention. In some embodiments, the treatment comprises causing the death of a category of cells, such as senescent cells, SASP cells, or CSCs likely to be involved in metastasis or recurrence, of a particular cancer in a host, and may be accomplished through preventing senescent cells and/or cancer cells from further propagation, and/or inhibiting CSC function through, for example, depriving such cells of mechanisms for generating energy. For example, treatment can be diminishment of one or several symptoms of a cancer, or complete eradication of a cancer. As another example, the present approach may be used to inhibit mitochondrial metabolism in the cancer, eradicate (e.g., killing at a rate higher than a rate of propagation) CSCs in the cancer, eradicate TICs in the cancer, eradicate circulating tumor cells in the cancer, inhibit propagation of the cancer, target and inhibit CSCs, target and inhibit TICs, target and inhibit circulating tumor cells, prevent or reduce the likelihood of, metastasis, prevent recurrence, sensitize the cancer to a chemotherapeutic, sensitize the cancer to radiotherapy, sensitize the cancer to phototherapy. As another example, the treatment can reduce the accumulated senescent cells, and/or reduce the rate of senescent cell accumulation.

In the context of tumor recurrence and/or metastasis, the term “prevent” and “reduce the likelihood of” refer to reducing, in a subject, the presence of CSCs, TICs, and circulating tumor cells, likely to be involved in recurrence or metastasis, to a level at which tumor recurrence and/or metastasis from the primary site is unlikely, relative to a control (i.e., no treatment to prevent or reduce the likelihood of tumor recurrence and/or metastasis). In practice, a treatment to prevent and/or reduce the likelihood of tumor recurrence and/or metastasis as described herein targets and inhibits or eradicates CSCs, TICs, inhibit circulating tumor cells.

The terms “cancer stem cell” and “CSC” refer to the subpopulation of cancer cells within tumors that have capabilities of self-renewal, differentiation, and tumorigenicity when transplanted into an animal host. Compared to “bulk” cancer cells, CSCs have increased mitochondrial mass, enhanced mitochondrial biogenesis, and higher activation of mitochondrial protein translation. As used herein, a “circulating tumor cell” is a cancer cell that has shed into the vasculature or lymphatics from a primary tumor and is carried around the body in the blood circulation. The CellSearch Circulating Tumor Cell Test may be used to detect circulating tumor cells.

The phrase “pharmaceutically effective amount,” as used herein, indicates an amount necessary to administer to a host, or to a cell, tissue, or organ of a host, to achieve a therapeutic result, such as regulating, modulating, or inhibiting protein kinase activity, e.g., inhibition of the activity of a protein kinase, or treatment of cancer. A physician or veterinarian having ordinary skill in the art can readily determine and prescribe the effective amount of the pharmaceutical composition required for a given subject, using methods well-known and available in the art. For example, the physician or veterinarian could start doses of the compounds of the invention employed in the pharmaceutical composition at levels lower than that required in order to achieve the desired therapeutic effect and gradually increase the dosage until the desired effect is achieved. The determination of a pharmaceutically effective amount is deemed to be within the purview of the person having an ordinary level of skill in the art, having reviewed this disclosure.

As used herein, the phrase “therapeutic agent” refers to an embodiment of the compound described herein, which may include a pharmaceutically acceptable salt or isotopic analog thereof. It should be appreciated that the therapeutic agent may be administered to the subject through any suitable approach, as would be known to those having an ordinary level of skill in the art. It should also be appreciated that the amount of therapeutic agent and the timing of its administration may be dependent on the individual subject being treated (e.g., the age and body mass, among other factors), on the manner of administration, on the pharmacokinetic properties of the particular therapeutic agent, and on the judgment of the prescribing physician. Thus, because of subject to subject variability, any dosages described herein are intended to be initial guidelines, and the physician can titrate doses of the therapeutic agent to achieve the treatment that the physician considers appropriate for the subject. In considering the degree of treatment desired, the physician can balance a variety of factors such as age and weight of the subject, presence of preexisting disease, as well as presence of other diseases. Pharmaceutical formulations can be prepared for any desired route of administration including, but not limited to, oral, intravenous, or aerosol administration, as discussed in greater detail below.

The phrase “pharmaceutically acceptable carrier” as used herein, means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose: (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The phrase “pharmaceutically acceptable salt” to the relatively non-toxic, inorganic and organic base addition salts of the compounds of the present approach. A pharmaceutically acceptable salt may be formed by, for example, reacting a compound in its free acid form with a base, such as hydroxide or carbonate of a pharmaceutically-acceptable metal cation, with ammonia or with a pharmaceutically-acceptable amine. Representative alkali or alkaline earth salts include sodium, potassium, calcium, magnesium, and aluminum salts, for example. Examples of amines that may be used for base addition salts include ethylamine, diethylamine, ethylenediamine, ethanolamine, diethanolamine, piperazine. It should be appreciated that other salts may be used, of course, and that the person of ordinary skill in the art may use methods known in the art for identifying suitable salt forms, without departing from the present approach.

As used herein, a “fatty acid” moiety is a carboxylic acid with an aliphatic chain, which is either saturated or unsaturated. Examples of fatty acids include short chain fatty acids (i.e., having 5 or fewer carbon atoms in the chemical structure), medium-chain fatty acids (having 6-12 carbon atoms in the chemical structure), and other long chain fatty acids (i.e., having 13-21 carbon atoms in the chemical structure). Examples of saturated fatty acids include lauric acid (CH₃(CH₂)₁₀COOH), palmitic acid (CH₃(CH₂)₁₄COOH), stearic acid (CH₃(CH₂)₁₆COOH), and myristic acid (CH₃(CH₂)₁₂COOH). Oleic acid (CH3(CH2)7CH═CH(CH2)7COOH) is an example of a naturally occurring unsaturated fatty acid. It should be appreciated that in some embodiments, compounds of the present approach include a linear, saturated fatty acid or amide group, and preferably a linear, saturated fatty acid or amide group having from 5 to 18 carbon atoms, and more preferably from 10 to 16 carbon atoms, and even more preferably, from 12 to 15 carbon atoms. In some of the preferred embodiments, the linear, saturated fatty acid is myristic acid, or the amide is tetradecanamide, having 14 carbon atoms.

Recent development identified a first-in-class inhibitor of mitochondrial transcription targeting mitochondrial RNA polymerase (POLRMT), a gene product responsible for mitochondrial gene expression and mitochondrial biogenesis of the OXPHOS system. The inhibitor has been used in a four-week trial in mice. The oral treatment with the compound showed no evidence of OXPHOS dysfunction or toxicity in the mice, but showed strong anti-tumor effects in human cancer cell xenografts.

Embodiments of the compounds disclosed herein may be used as therapeutic agent to selectively eradicate CSCs for treating and/or preventing tumor recurrence and/or metastasis. The data demonstrates that the compounds disclosed herein have anti-cancer activity, and are suitable for use as therapeutic agents for anti-cancer treatments, including treating and/or preventing tumor recurrence and metastasis. Data described herein demonstrates the anti-cancer activity through inhibition of MCF7 cells via the mammosphere formation assay, and also cell viability using the sulforhodamine B assay (also known in the art and referred to herein as the SRB assay). This assay measures the amount of residual protein that adheres to tissue culture dishes and is a surrogate marker of cell viability.

Under the present approach, embodiments of the therapeutic compound may have the generic chemical structure of Formula 1, shown below:

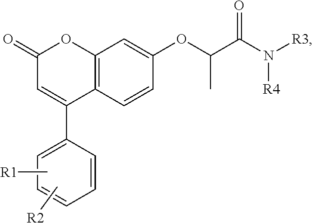

or pharmaceutically acceptable salts thereof, in which:
R1 and R2 may be the same or different, and may be a halogen, CF₂H, —CF₃, —OCF₂H, —OCF₃, substituted or unsubstituted C5-C18 carboxyl, substituted or unsubstituted C5-C18 alkane, substituted or unsubstituted C5-C18 alkene, substituted or unsubstituted C5-C18 cyclic alkene, substituted or unsubstituted C5-C18 alkyne, substituted or unsubstituted C5-C18 ketone, substituted or unsubstituted C5-C18 aldehyde, substituted or unsubstituted C5-C18 ether, substituted or unsubstituted C5-C18 ester, substituted or unsubstituted C5-C18 amine, substituted or unsubstituted C5-C18 amide, substituted or unsubstituted C5-C18 alkyl-amide, monocyclic or polycyclic arene, heteroarene, phenol, or benzoic acid; and
R3 and R4 may be the same or different, and may be hydrogen, substituted or unsubstituted C2-C18-alkyl, substituted or unsubstituted C3-C8-cycloalkyl, substituted or unsubstituted pyridine, substituted or unsubstituted C2-C18 carboxyl, substituted or unsubstituted C2-C18 alkene, substituted or unsubstituted C2-C18 alkyne, substituted or unsubstituted C2-C18 ketone, substituted or unsubstituted C2-C18 aldehyde, substituted or unsubstituted C2-C18 ether, substituted or unsubstituted C2-C18 ester, substituted or unsubstituted C2-C18 amine, substituted or unsubstituted C2-C18 amide, substituted or unsubstituted C2-C18 alkyl-amide, substituted or unsubstituted phenol, or benzoic acid, or one of R3 and R4 is the group

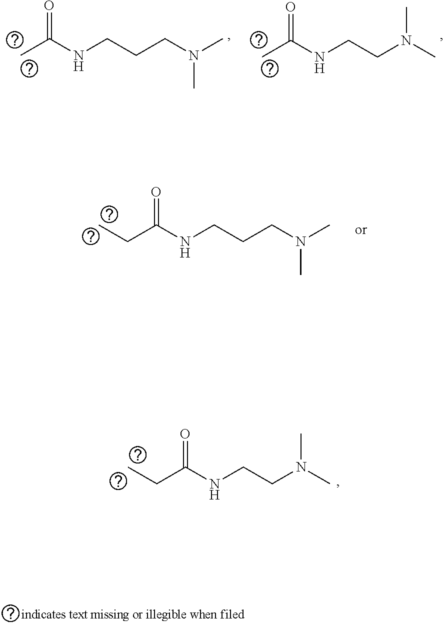

and the other is a H or a C2-C5 alkyl; or R3 and R4 form a substituted or unsubstituted C5 or C6 heterocycle; or R3 and R4 form the group

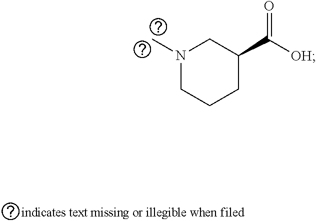

or NR3R4 is OH;
provided that at least one of R1 and R2 is a substituted or unsubstituted C5-C18 amide, or at least one of R3 and R4 is selected from the group consisting of:

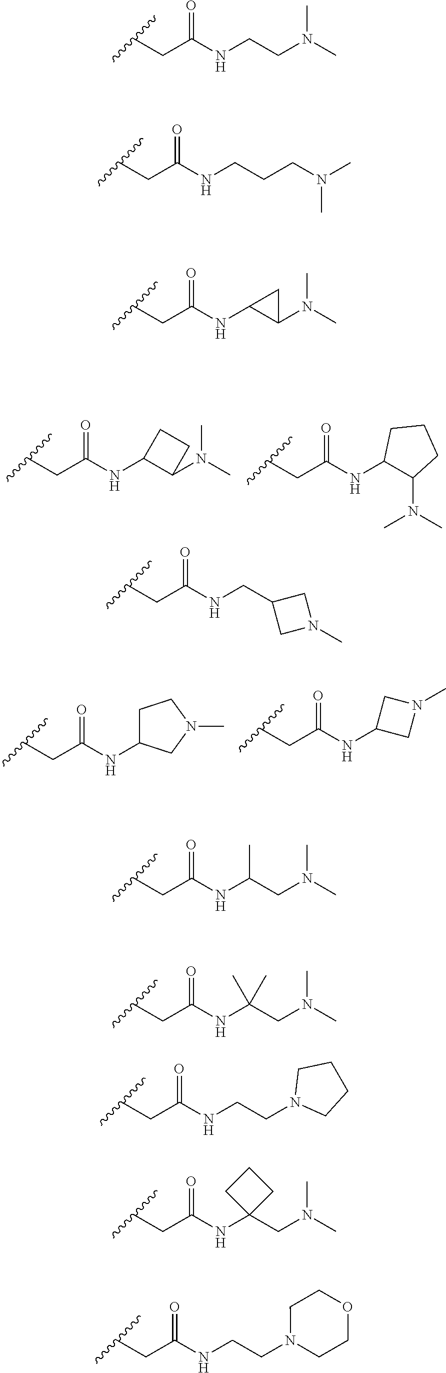

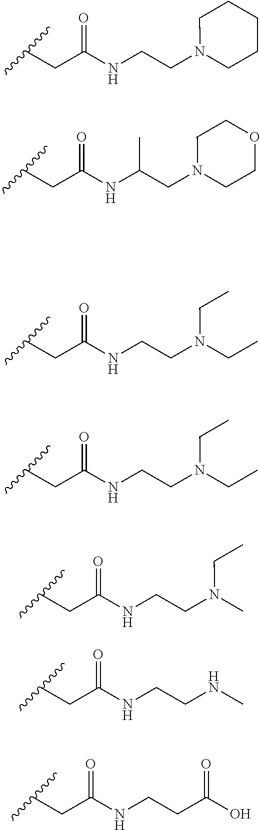

and the other is H or a C2-C5 alkyl.
   In some preferred embodiments, at least one of R1 and R2 is a substituted or unsubstituted C5-C18 amide, or at least one of R3 and R4 is the group

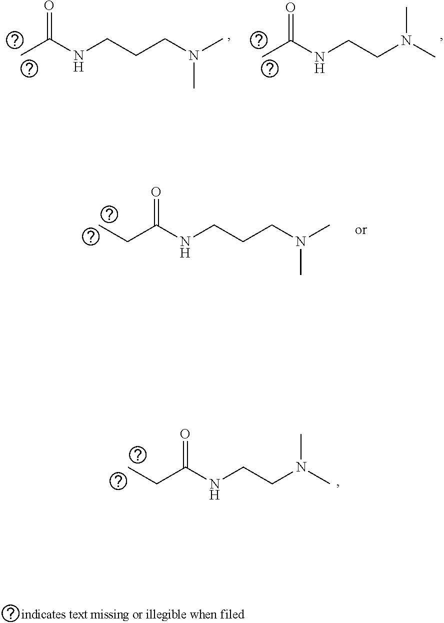

and the other is a H or a C2-C5 alkyl.

Compounds having the generic structure of Formula 1 may be prepared according to the following general reaction scheme, in which a substituted benzoic acid may be used as the initial compound. Functional groups R1, R2, R3, and R4 may be as described above.

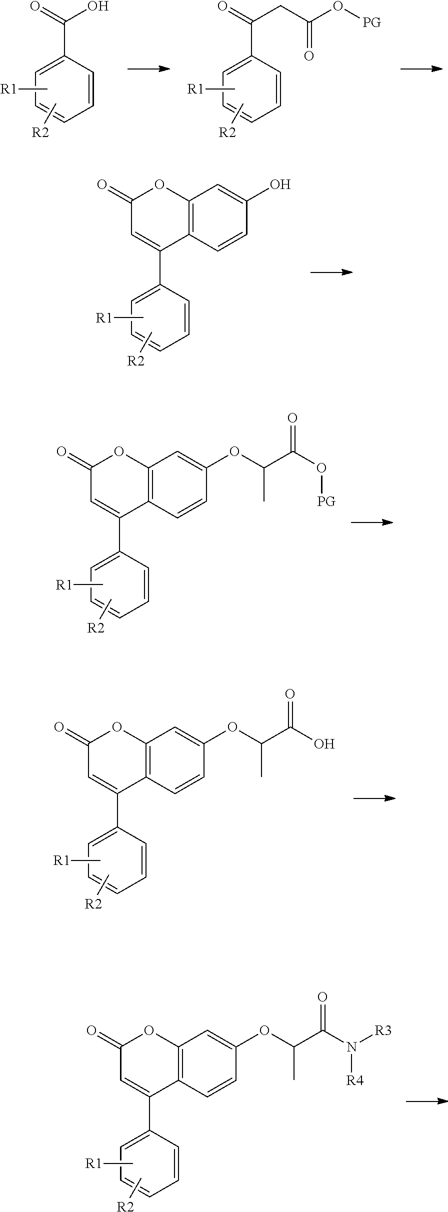

It should be appreciated that other synthesis methods may be used to arrive at the compounds of the present approach.

The following compounds are examples of therapeutic agents according to the present approach, in which R1 is a halogen, and R2 is an alkyl amide group. It should be appreciated that R1 and R2 may occupy other positions on the phenyl group without departing from the present approach.

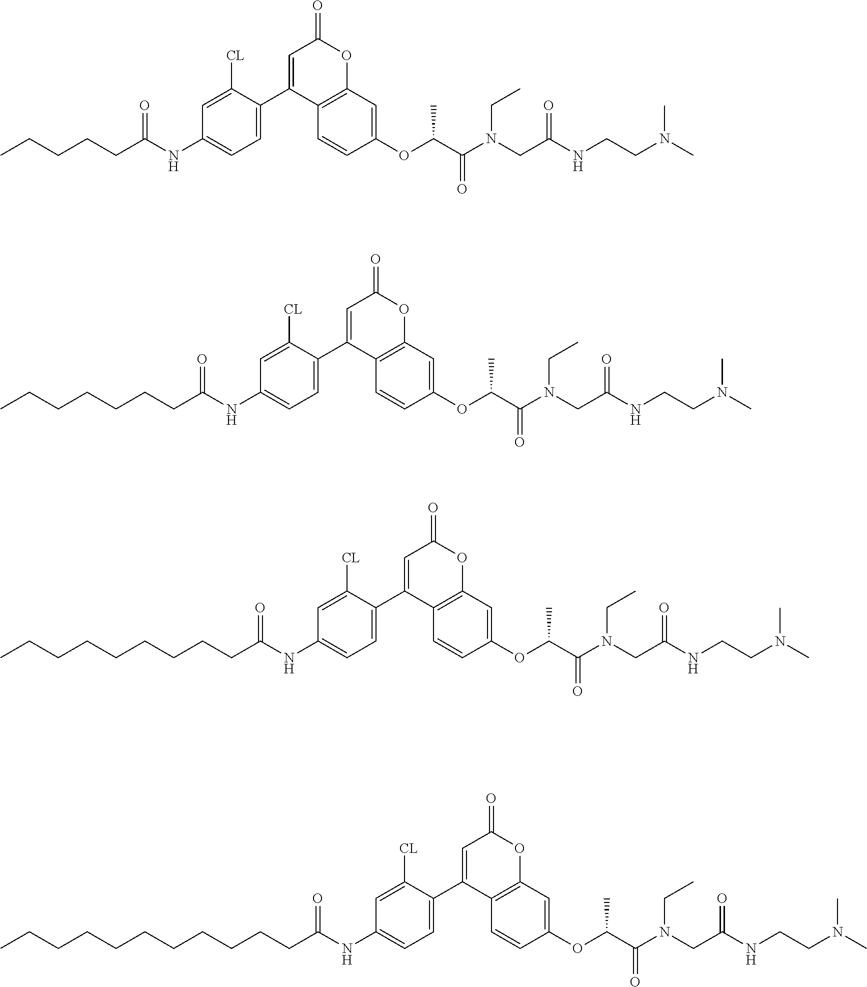

The following compounds are examples of therapeutic agents according to the present approach, demonstrating variations in R3 and R4, and also various enantiomers. As the person having an ordinary level of skill in the art would recognize, R1 and R2 reference functional groups, and the ‘R’ and ‘S’ symbols indicate enantiomers at the stereocenter for the particular embodiment.

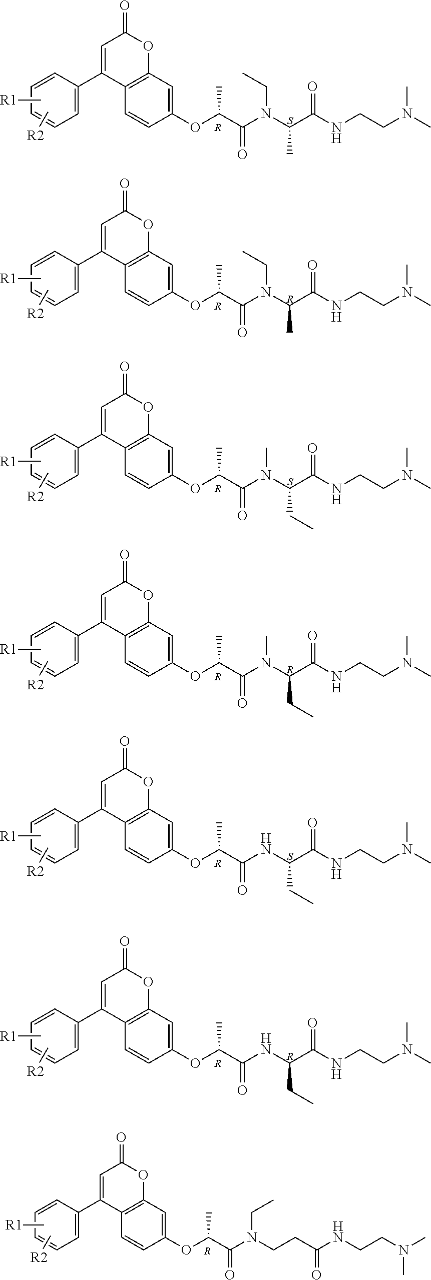

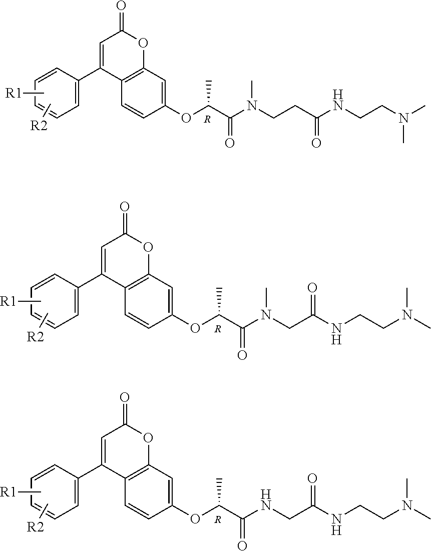

Compounds having the generic structure of Formula 1, in which R1 is a halogen (e.g., chloro or fluoro), and R2 is an alkyl amide, may be prepared according to the following general reaction scheme, in which a substituted benzoic acid may be used as the initial compound. Functional groups R1, R2, R3, and R4 may be as described above, and specific examples are described below.

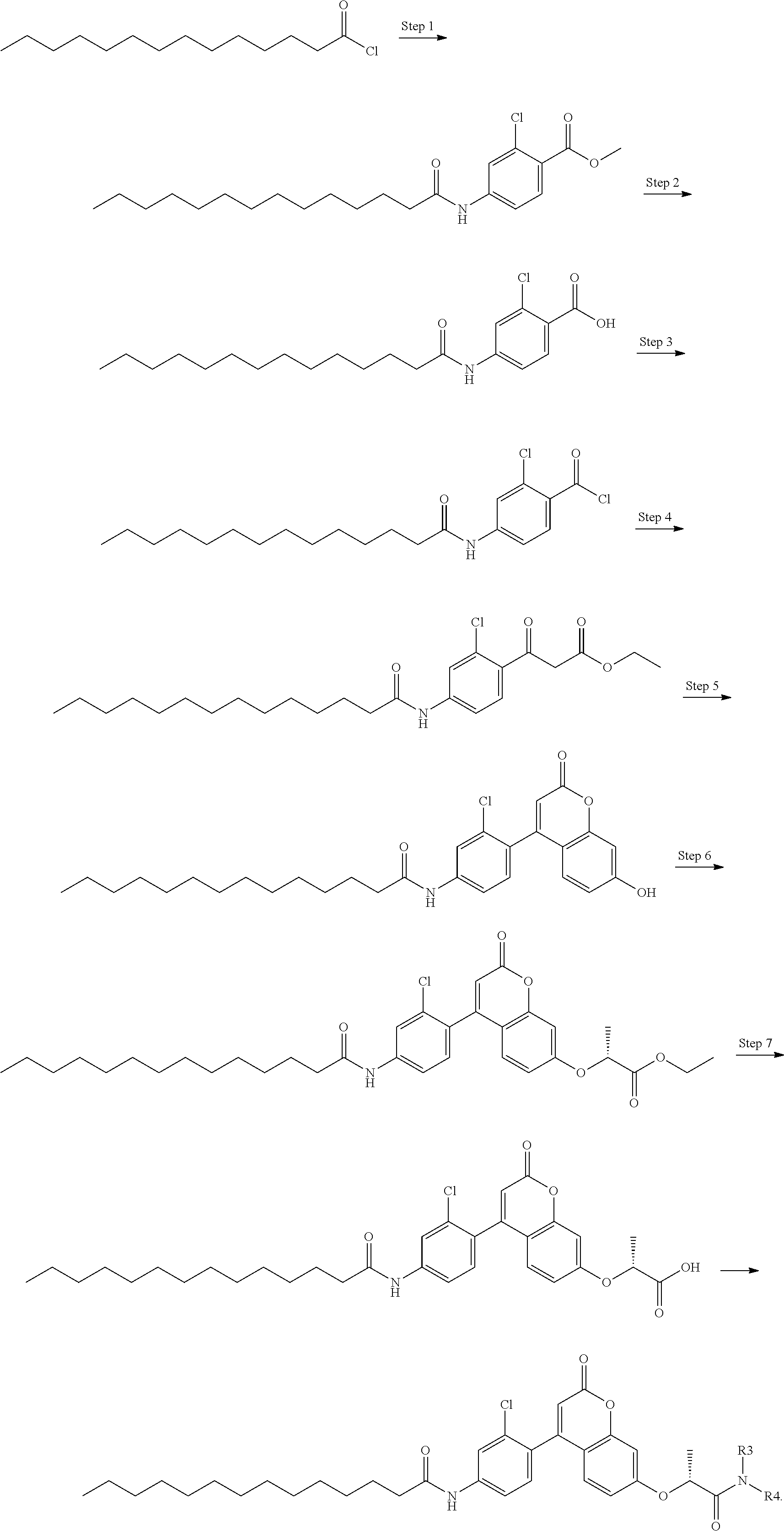

It should be appreciated that the halogen and the alkyl amide may occupy different positions on the phenyl, other than as illustrated in the above example.

As another example, some embodiments of the therapeutic agents described herein may be prepared according to the reaction scheme below.

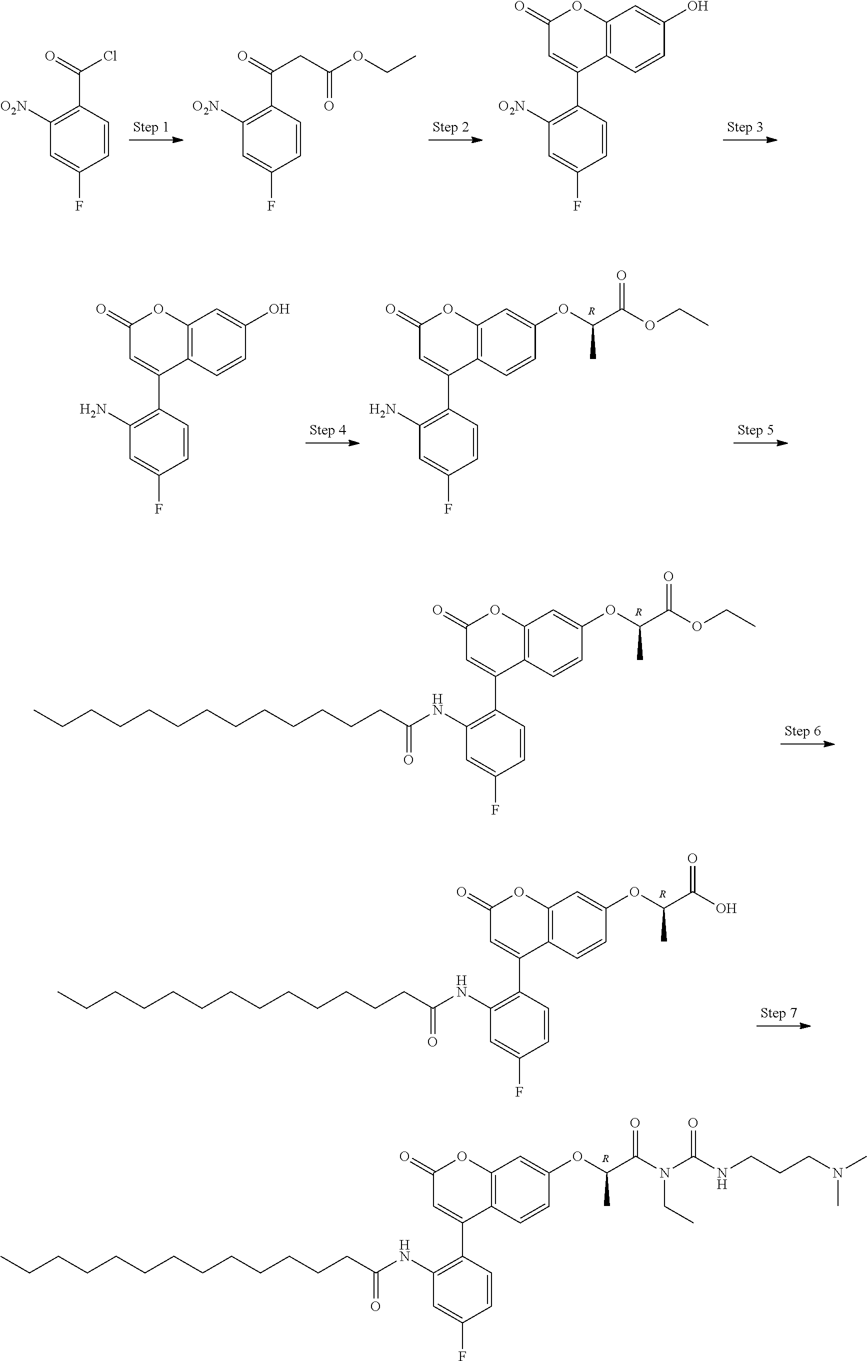

In this illustrated embodiment, R1 is fluoro, R2 is an alkyl amide, R3 is ethyl, and R4 is the (dimethylamino)propyl amide. As with other example embodiments described herein, it should be appreciated that the halogen and the alkyl amide may occupy different positions on the phenyl, other than as illustrated in the above example.

Compounds of the present approach show significantly improved therapeutic activity relative to Compound [I], having the chemical structure shown below:

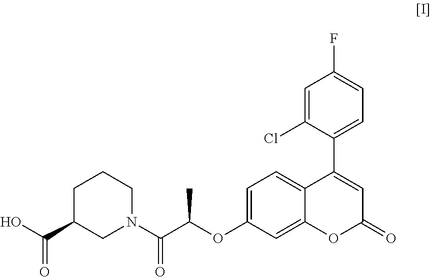 [SMILES: C[C@@H](Oc1ccc2c(-c3ccc(F)cc3Cl)cc(=O)oc2c1)C(=O)N1CCC[C@H](C(=O)O)C1]

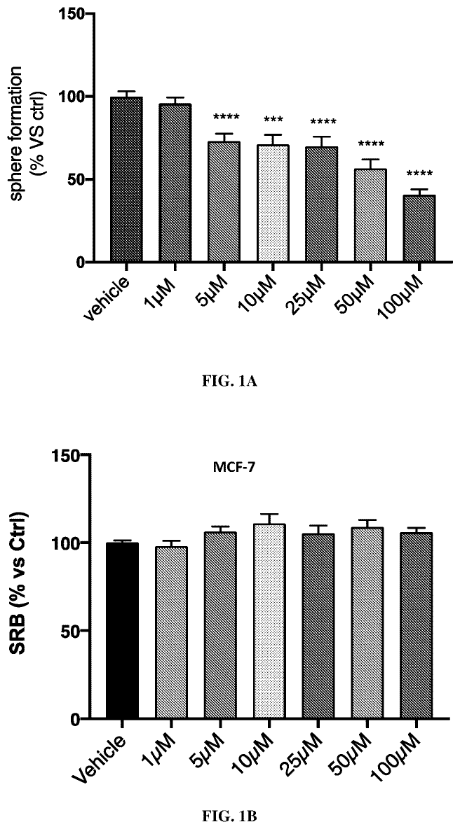

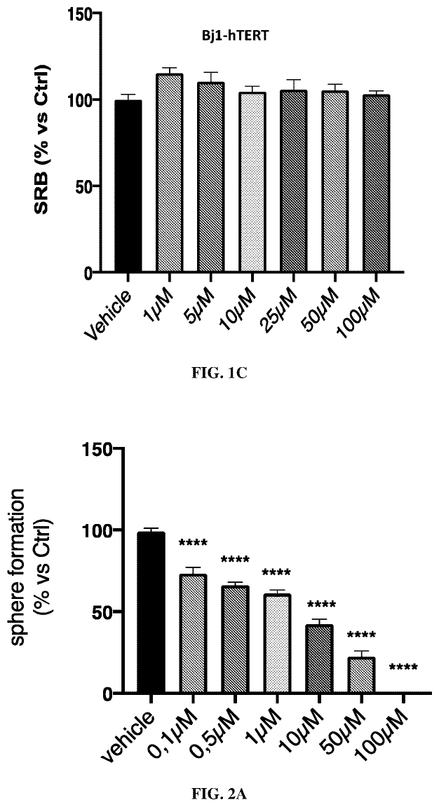

Compound [I], 3-piperidinecarboxylic acid, 1-[(2R)-2-[[4-(2-chloro-4-fluorophenyl)-2-oxo-2H-1-benzopyran-7-yl]oxy]-1-oxopropyl]-, (3S)- (CAS No.: 2304621-06-3), has an IC₅₀ of approximately 50 μM against MCF-7 cells. FIGS. 1A-1C show mammosphere formation results, SRB assay results on MCF-7 cells, and SRB assay results on BJ1-hTERT cells, for Compound [I]. As can be seen from FIG. 1A, Compound [I] showed inhibition of MCF-7 cells at a concentration of 5 μM, and achieved greater than 50% inhibition at a concentration of 100 μM. FIGS. 1B and 1C show that Compound [I] did not exhibit cytotoxicity towards MCF-7 or BJ1-hTERT cells at all tested concentrations between 1 μM and 100 μM.

In some embodiments, the therapeutic agent may take the form of Compound [II], (3S)-1-[(2R)-2-[4-[2-Chloro-4-(tetradecanoylamino)phenyl]-2-oxo-chromen-7-yl]oxypropanoyl]piperidine-3-carboxylic acid, having the chemical structure shown below:

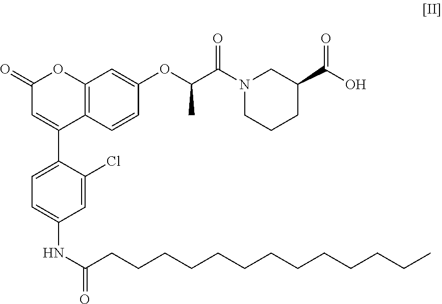 [SMILES: CCCCCCCCCCCCCC(=O)Nc1ccc(-c2cc(=O)oc3cc(O[C@H](C)C(=O)N4CCC[C@H](C(=O)O)C4)ccc23)c(Cl)c1]

Compound II, (3S)-1-[(2R)-2-[4-[2-Chloro-4-(tetradecanoylamino)phenyl]-2-oxo-chromen-7-yl]oxypropanoyl]piperidine-3-carboxylic acid, may be synthesized according to the following process. In Step 1, ethyl (3S)-1-[(2R)-2-[4-[2-chloro-4-(tetradecanoylamino)phenyl]-2-oxo-chromen-7-yl]oxypropanoyl]piperidine-3-carboxylate was prepared as an intermediate. To a stirred solution of (2R)-2-[4-[2-chloro-4-(tetradecanoylamino)phenyl]-2-oxo-chromen-7-yl]oxypropanoic acid (147 mg, 0.25 mmol) and (S)-ethyl piperidine-3-carboxylate in DMA (1 ml) at room temperature under nitrogen atmosphere NMM (35.5 μl, 0.32 mmol) and EDC hydrochloride (62 mg, 0.32 mmol) was added. The mixture was stirred for 16 hours, diluted with EtOAc (40 ml), washed with 2M HCl (15 ml), water (15 ml), saturated NaHCO₃ (15 ml), brine (15 ml), dried over MgSO₄, filtered and the filtrate was evaporated under reduced pressure to yield a crude product. Purification on silica gel (30% EtOAc in iso-hexane) afforded ethyl (3S)-1-[(2R)-2-[4-[2-chloro-4-(tetradecanoylamino)phenyl]-2-oxo-chromen-7-yl]oxypropanoyl]piperidine-3-carboxylate, (19.6 mg). LC-MS 709.4 [M+1]⁺, RT 6.86 min.

In Step 2, (3S)-1-[(2R)-2-[4-[2-Chloro-4-(tetradecanoylamino)phenyl]-2-oxo-chromen-7-yl]oxypropanoyl]piperidine-3-carboxylic acid was prepared from the intermediate. To a stirred solution of ethyl (3S)-1-[(2R)-2-[4-[2-chloro-4-(tetradecanoylamino)phenyl]-2-oxo-chromen-7-yl]oxypropanoyl]piperidine-3-carboxylate (19 mg, 0.027 mmol) in THF (1.5 ml) and MeOH (0.5 ml) at room temperature a solution of 1M NaOH (75 μl, 0.075 mmol) was added and the mixture was stirred for 16 hours. The solvent was evaporated under reduced pressure, the residue was suspended in EtOAc (30 ml), acidified with 2M HCl (10 ml), the extract was washed with brine (10 ml), dried over MgSO₄, the solid residue was removed by filtration and the solvent was evaporated under reduced pressure to yield (3S)-1-[(2R)-2-[4-[2-Chloro-4-(tetradecanoylamino)phenyl]-2-oxo-chromen-7-yl]oxypropanoyl]piperidine-3-carboxylic acid as a solid, (17.3 mg). LC-MS 681.4 [M+1]⁺, RT 6.39 min.

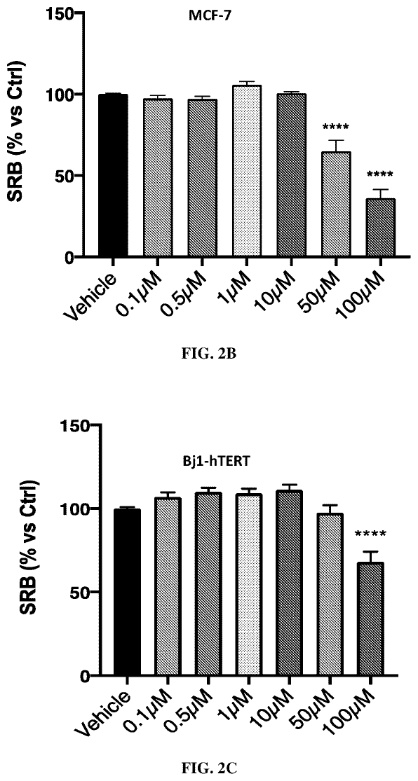

FIGS. 2A-2C show mammosphere formation results, SRB assay results on MCF-7 cells, and SRB assay results on BJ1-hTERT cells, for Compound [II]. As can be seen from FIG. 2A, Compound [II] showed a dose-dependent inhibition of MCF-7 cells at all tested concentrations, ranging from 0.1 μM to 100 μM. Compound [II] achieved greater than 50% inhibition at a concentration of 10 μM, and a 100% inhibition at 100 μM. With an IC50 of approximately 5 μM, Compound [II] is 10 times as potent as Compound [I] with respect to inhibiting MCF-7 mammosphere formation. FIGS. 2B and 2C show that Compound [II] exhibits cytotoxicity towards MCF-7 and BJ1-hTERT cells starting at 50 μM.

In some embodiments, the therapeutic agent may take the form of Compound [III], tert-Butyl-N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]carbamate, having the chemical structure shown below:

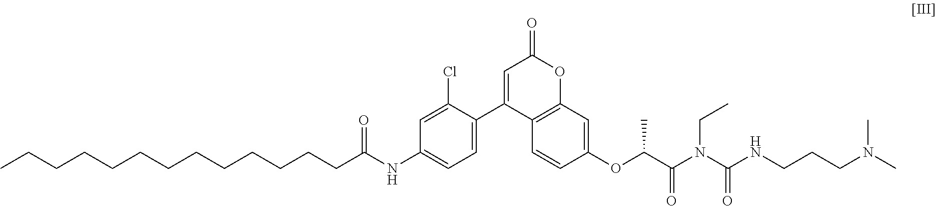 [SMILES: CCCCCCCCCCCCCC(=O)Nc1ccc(-c2cc(=O)oc3cc(O[C@H](C)C(=O)N(CC)C(=O)NCCCN(C)C)ccc23)c(Cl)c1]

Compound [III] was prepared from 2-chloro-4-nitrobenzoic acid following the method in Advance Synthesis and Catalysis, 2014, vol. 356, no. 11-12, p 2437-2444, a yielding crude 2-chloro-4-nitro-benzoyl chloride as a light brown oil (11.78 g). Ethyl 3-(2-chloro-4-nitro-phenyl)-3-oxo-propanoate was then prepared from the crude 2-chloro-4-nitro-benzoyl chloride following the method in Step 1 for Compound [IV], affording ethyl 3-(2-chloro-4-nitro-phenyl)-3-oxo-propanoate as a light brown solid (11.04 g). LC-MS 272.0 [M+1]⁺, RT 5.39 min.

Next, 4-(2-chloro-4-nitro-phenyl)-7-hydroxy-chromen-2-one was prepared from the ethyl 3-(2-chloro-4-nitro-phenyl)-3-oxo-propanoate following the method in Step 2 of Compound [IV], affording 4-(2-chloro-4-nitro-phenyl)-7-hydroxy-chromen-2-one as a purple solid (5.85 g). LC-MS 318.0 [M+1]⁺, RT 5.18 min.

Next, 4-(4-amino-2-chloro-phenyl)-7-hydroxy-chromen-2-one was prepared from 4-(2-chloro-4-nitro-phenyl)-7-hydroxy-chromen-2-one the following the method in Step 3 of Compound [IV], affording 4-(4-amino-2-chloro-phenyl)-7-hydroxy-chromen-2-one as a dark red-brown solid (3.02 g). LC-MS 287.8 [M+1]⁺, RT 4.43 min.

The 4-(4-amino-2-chloro-phenyl)-7-hydroxy-chromen-2-one was used to prepare ethyl (2R)-2-[4-(4-amino-2-chloro-phenyl)-2-oxo-chromen-7-yl]oxypropanoate, following the method in Step 4 of Compound [IV], resulting in ethyl (2R)-2-[4-(4-amino-2-chloro-phenyl)-2-oxo-chromen-7-yl]oxypropanoate as a light brown solid (1.57 g). LC-MS 388.0 [M+1]⁺, RT 5.27 min.

Ethyl (2R)-2-[4-[4-(tert-butoxycarbonylamino)-2-chloro-phenyl]-2-oxo-chromen-7-yl]oxypropanoate was next prepared. To a stirred solution of ethyl (2R)-2-[4-(4-amino-2-chloro-phenyl)-2-oxo-chromen-7-yl]oxypropanoate (0.26 g, 0.70 mmol) and DMAP (0.21 g, 1.70 mmol) in DCM (20 ml) at room temperature under nitrogen atmosphere Boc-anhydride (0.37 g, 1.70 mmol) was added. The mixture was stirred for 16 hours and the solvent was evaporated under reduced pressure to yield a crude product. The residue was dissolved in EtOAc (40 ml), washed with 2M HCl (20 ml), saturated NaHCO₃ (20 ml), brine (20 ml), dried over MgSO₄, filtered and the solvent was evaporated under reduced pressure to yield a crude product. Trituration with n-pentane afforded ethyl (2R)-2-[4-[4-(tert-butoxycarbonylamino)-2-chloro-phenyl]-2-oxo-chromen-7-yl]oxypropanoate (0.54 g). LC-MS 488.0 [M+1]⁺, RT 6.60 min.

As the next step, (2R)-2-[4-[4-(tert-Butoxycarbonylamino)-2-chloro-phenyl]-2-oxo-chromen-7-yl]oxypropanoic acid was prepared from ethyl (2R)-2-[4-[4-(tert-butoxycarbonylamino)-2-chloro-phenyl]-2-oxo-chromen-7-yl]oxypropanoate the following the method in Step 6 of Compound [IV], affording (2R)-2-[4-[4-(tert-butoxycarbonylamino)-2-chloro-phenyl]-2-oxo-chromen-7-yl]oxypropanoic acid as a light brown solid (0.29 g). LC-MS 460.0 [M+1]⁺, RT 5.98 min.

Finally, tert-butyl N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]carbamate was prepared from (2R)-2-[4-[4-(tert-butoxycarbonylamino)-2-chloro-phenyl]-2-oxo-chromen-7-yl]oxypropanoic acid the following the method in Example 6 Step 3 affording tert-butyl N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]carbamate (structure shown below, where “Boc” represents tert-butyloxycarbonyl) as a light brown solid (0.15 g). LC-MS 615.2 [M+1]⁺, RT 4.38 min.

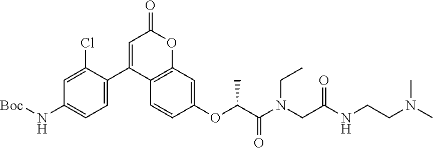 [SMILES: CCN(CC(=O)NCCN(C)C)C(=O)[C@@H](C)Oc1ccc2c(-c3ccc(NC(=O)OC(C)(C)C)cc3Cl)cc(=O)oc2c1]

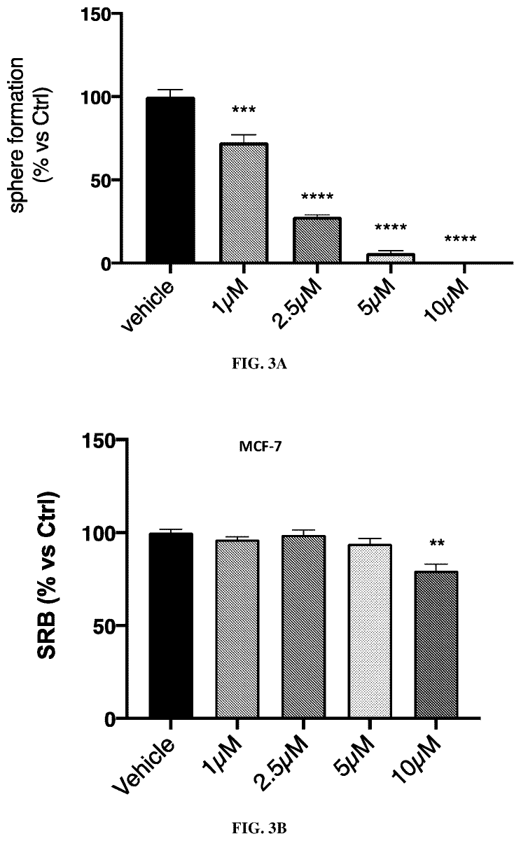

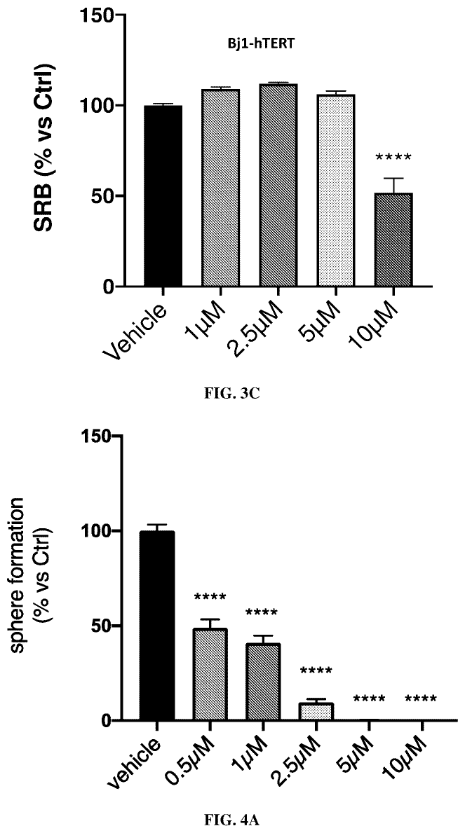

FIGS. 3A-3C show mammosphere formation results, SRB assay results on MCF-7 cells, and SRB assay results on BJ1-hTERT cells, for Compound [III]. As can be seen from FIG. 3A, Compound [III] showed a dose-dependent inhibition of MCF-7 cells at all tested concentrations, ranging from 1 μM to 10 μM. The IC50 of Compound [III] is between 1 μM and 2.5 μM, and at a concentration of 10 μM, a twenty-fold improvement relative to Compound [I]. Further, Compound [III] achieved 100% inhibition of MCF-7 cells. FIGS. 2B and 2C show that Compound [III] exhibits cytotoxicity towards MCF-7 and BJ1-hTERT cells starting at 10 μM.

In some embodiments, the therapeutic agent may take the form of Compound [IV], N-[2-[7-[(1R)-2-[3-(Dimethylamino)propylcarbamoyl-ethyl-amino]-1-methyl-2-oxo-ethoxy]-2-oxo-chromen-4-yl]-5-fluoro-phenyl]tetradecanamide, having the chemical structure shown below:

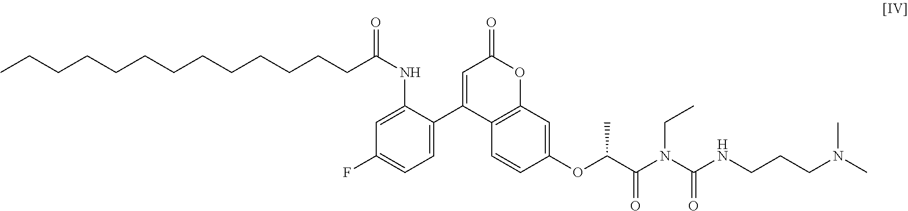 [SMILES: CCCCCCCCCCCCCC(=O)Nc1cc(F)ccc1-c1cc(=O)oc2cc(O[C@H](C)C(=O)N(CC)C(=O)NCCCN(C)C)ccc12]

Compound [IV] was prepared from using Compound [VII], (2R)-2-[4-[4-fluoro-2-(tetradecanoylamino)phenyl]-2-oxo-chromen-7-yl]oxypropanoic acid, as an intermediary, the synthesis of which is described below. To a stirred solution of (2R)-2-[4-[4-fluoro-2-(tetradecanoylamino)phenyl]-2-oxo-chromen-7-yl]o2xypropanoic acid (60 mg, 0.10 mmol) in DMA (4 ml) at room temperature under nitrogen atmosphere NMM (23 μl, 0.21 mmol) and EDC hydrochloride (27 mg, 0.14 mmol) was added. The mixture was stirred for 16 hours and the solvent was evaporated under reduced pressure to yield a crude product. Purification on silica gel (2.5% MeOH in DCM) afforded N-[2-[7-[(1R)-2-[3-(dimethylamino)propylcarbamoyl-ethyl-amino]-1-methyl-2-oxo-ethoxy]-2-oxo-chromen-4-yl]-5-fluoro-phenyl]tetradecanamide, (36.8 mg, 52%). LC-MS 709.6 [M+1]⁺, RT 5.14 min.

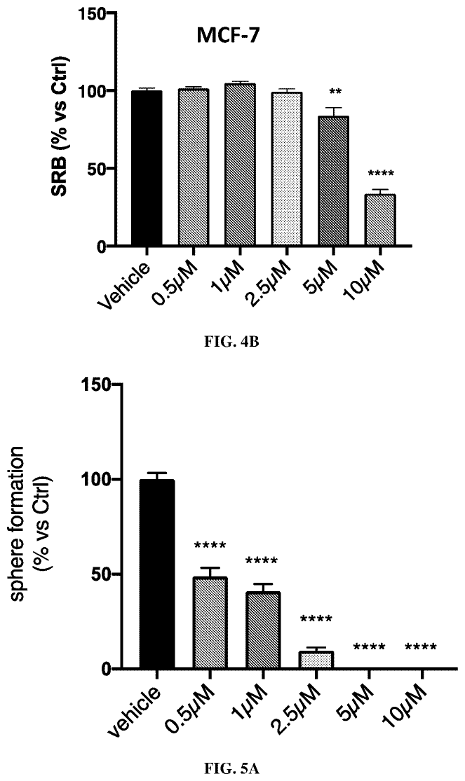

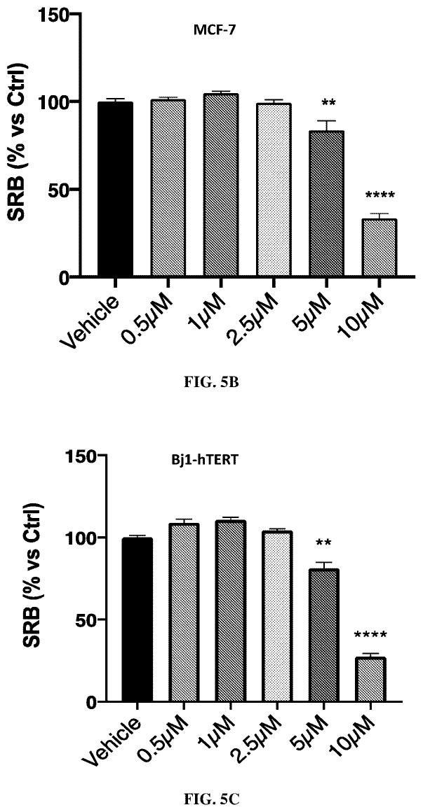

FIGS. 4A and 4B show mammosphere formation results and SRB assay results on MCF-7 cells, for Compound [IV]. FIG. 4A shows that Compound [IV] has a dose-dependent inhibition of MCF-7 cells at all tested concentrations, ranging from 1 μM to 10 μM. Compound [IV] showed over 50% inhibition at 2.5 μM, and near complete inhibition at 10 μM. FIG. 4B shows that Compound [IV] exhibits cytotoxicity towards MCF-7 T cells starting at 5 μM.

In some embodiments, the therapeutic agent may take the form of Compound [V], N-[3-Chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]tetradecanamide, having the chemical structure shown below:

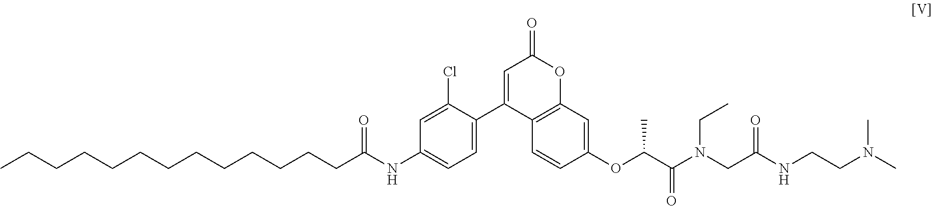 [SMILES: CCCCCCCCCCCCCC(=O)Nc1ccc(-c2cc(=O)oc3cc(O[C@H](C)C(=O)N(CC)CC(=O)NCCN(C)C)ccc23)c(Cl)c1]

Compound [V] was synthesized as follows. In Step 1, tert-butyl N-[2-[2-(dimethylamino)ethylamino]-2-oxo-ethyl]-N-ethyl-carbamate was produced as an intermediate. To a stirred solution of Boc-N-ethylglycine (1.02 g, 5.0 mmol) and NMM (0.79 ml, 7.5 mmol) in DCM (30 ml) at 0° C. under nitrogen atmosphere iso-butyl chloroformate (0.82 ml, 6.0 mmol) was added. The mixture was stirred for 1 hour, N,N-dimethyl ethylenediamine (0.83 ml, 7.5 mmol) was added, the mixture was allowed to warm to room temperature and stirred for 60 hours. The reaction mixture diluted with DCM (30 ml), washed with saturated NaHCO₃ (30 ml), brine (30 ml), dried over MgSO₄, filtered and the filtrate was evaporated under reduced pressure to yield tert-butyl N-[2-[2-(dimethylamino)ethylamino]-2-oxo-ethyl]-N-ethyl-carbamate as a light brown oil, (1.44 g). LC-MS 274.3 [M+1]⁺, RT 0.86 min.

In Step 2, N-[2-(dimethylamino)ethyl]-2-(ethylamino)acetamide hydrochloride was prepared. To a stirred solution of tert-butyl N-[2-[2-(dimethylamino)ethylamino]-2-oxo-ethyl]-N-ethyl-carbamate (1.30 g, 4.8 mmol) in chloroform (30 ml) at room temperature under nitrogen atmosphere 4M HCl in dioxane (23 ml, 95 mmol) was added and the mixture was stirred for 20 hours. The solvent was evaporated under reduced pressure to yield N-[2-(dimethylamino)ethyl]-2-(ethylamino)acetamide hydrochloride as a light brown solid, (1.15 g). LC-MS 174.3 [M+1]⁺, RT 0.27 min.

In Step 3, N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]tetradecanamide was prepared from the prior product. To a stirred solution of (2R)-2-[4-[2-chloro-4-(tetradecanoylamino)phenyl]-2-oxo-chromen-7-yl]oxypropanoic acid ((40 mg, 0.070 mmol) and NMM (23 μl, 0.21 mmol) in dry DCM (2 ml) at room temperature under nitrogen atmosphere iso-butyl chloroformate (11 μl, 0.084 mmol) was added. The mixture was stirred for 1 hour, N-[2-(dimethylamino)ethyl]-2-(ethylamino)acetamide hydrochloride (27 mg, 0.11 mmol) was added, the mixture was stirred for 17 hours. The reaction mixture was concentrated under reduced pressure, the residue was dissolved in EtOAc (20 ml), washed with water (10 ml), brine (10 ml), dried over MgSO₄, filtered and the filtrate was evaporated under reduced pressure. Purification on silica gel (5-10% MeOH in DCM with 1% 7M NH₃ in MeOH) afforded Compound [V], N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]tetradecanamide, (14.9 mg). LC-MS 725.6 [M+1]⁺, RT 5.35 min.

FIGS. 5A-5C show mammosphere formation results, SRB assay results on MCF-7 cells, and SRB assay results on BJ1-hTERT cells, for Compound [V]. FIG. 5A shows that Compound [V] effectively inhibited MCF-7 cells at all tested concentrations, ranging from 0.5 μM to 10 μM. Over 50% inhibition was seen at 0.5 μM, and at a concentration of 5 μM, Compound [V] achieved 100% inhibition of MCF-7 cells. FIGS. 5B and 5C show that Compound [V] exhibits cytotoxicity towards MCF-7 and BJ1-hTERT cells starting at 5 μM.

In some embodiments, the therapeutic agent may take the form of Compound [VI], (2R)-2-[4-[2-chloro-4-(tetradecanoylamino)phenyl]-2-oxo-chromen-7-yl]oxypropanoic acid, having the chemical structure shown below:

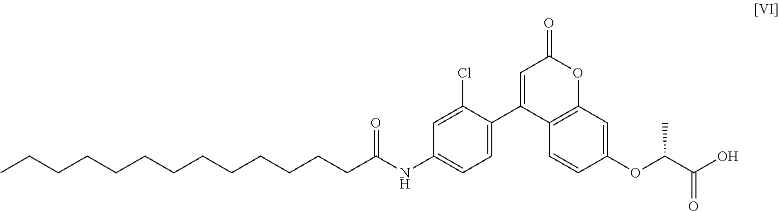 [SMILES: CCCCCCCCCCCCCC(=O)Nc1ccc(-c2cc(=O)oc3cc(O[C@H](C)C(=O)O)ccc23)c(Cl)c1]

Compound [VI] was synthesized according to the following approach, and it should be understood that alternative methods may be used.

In Step 1, methyl 2-chloro-4-(tetradecanoylamino)benzoate was produced as an initial intermediary. To a stirred suspension of methyl 4-amino-2-chlorobenzoate (1.87 g, 10.1 mmol) and NMM (1.50 ml, 13.6 mmol) in dry THF (30 ml) under nitrogen atmosphere at room temperature, tetradecanoyl chloride (2.42 g, 9.80 mmol) in dry DCM (10 ml) was added, and the mixture was stirred at room temperature for 20 hours. The solvent was evaporated under reduced pressure, the residue was dissolved in EtOAc (50 ml), washed with 2M HCl (50 ml) saturated NaHCO₃ (50 ml), brine (50 ml), dried over MgSO₄, filtered and the filtrate was concentrated under reduced pressure to yield a crude methyl 2-chloro-4-(tetradecanoylamino)benzoate as a light brown solid, (4.10 g). LC-MS 396.1 [M+1]⁺, RT 6.50 min.

In Step 2, the compound 2-chloro-4-(tetradecanoylamino)benzoic acid was prepared. To a stirred solution of methyl 2-chloro-4-(tetradecanoylamino)benzoate (3.60 g, 9.08 mmol) in THF (50 ml) and MeOH (50 ml) at room temperature a solution of NaOH (8 g 200 mmol) in water (50 ml) was added and the mixture was stirred for 16 hours. The solvent was evaporated under reduced pressure, the residue was acidified with 6M HCl (50 ml), diluted with water (50 ml) and the product was extracted with EtOAc (100 ml). The extract was washed with brine (ClH₂NaO, 50 ml), dried over MgSO₄, filtered and the solvent was evaporated under reduced pressure to yield a crude product, 2-chloro-4-(tetradecanoylamino)benzoic acid as a light yellow solid, (3.40 g). LC-MS 382.2 [M+1]⁺, RT 6.73 min.

In Step 3, 2-chloro-4-(tetradecanoylamino)benzoyl chloride was prepared. To a stirred suspension of 2-chloro-4-(tetradecanoylamino)benzoic acid (2.00 g, 5.24 mmol) and DMF (4 drops) in dry DCM (100 ml) under nitrogen atmosphere at room temperature oxalyl chloride (0.91 ml, 10.47 mmol) was added. The mixture was stirred at room temperature for 20 hours and concentrated under reduced pressure to yield a crude product, 2-chloro-4-(tetradecanoylamino)benzoyl chloride as an yellow solid, (2.30 g). LC-MS 396.3 [M-OMe+1]⁺, RT 7.21 min.

In Step 4, the compound ethyl 3-[2-chloro-4-(tetradecanoylamino)phenyl]-3-oxo-propanoate was prepared from 2-chloro-4-(tetradecanoylamino)benzoyl chloride as follows. To a stirred suspension of ethyl potassium malonate (1.9 g, 11.19 mmol) in dry MeCN (30 ml) under nitrogen atmosphere at +10° C. TEA (1.65 ml, 11.74 mmol) and anhydrous magnesium chloride (1.28 g, 13.42 mmol) was added, and the mixture was stirred at room temperature for 3.5 hours. A solution of 4-fluoro-2-nitrobenzoic acid chloride, prepared according to Archiv der Pharmazie, 1985, vol. 318, No. 1, p. 78-84, (1.14 g, 5.59 mmol) in dry MeCN (2 ml) was added and the mixture was stirred at room temperature for 16 hours. The reaction mixture was poured into a stirred mixture of saturated NH₄Cl (2 ml) and EtOAc (50 ml) and the solvent was evaporated under reduced pressure. The solid residue was stirred with 4M HCl (30 ml) at room temperature for 30 minutes and the product was extracted with EtOAc (50 ml). The extract was washed with 4m HCl (30 ml), water (30 ml), brine (30 ml), dried over MgSO₄, filtered and the filtrate was concentrated under reduced pressure to yield a crude product. Purification on silica gel (15% EtOAc in iso-hexane), resulting in ethyl 3-[2-chloro-4-(tetradecanoylamino)phenyl]-3-oxo-propanoate as an yellow solid, (2.10 g). LC-MS 452.2 [M+1]⁺, RT 7.05 min.

In Step 5, N-[3-chloro-4-(7-hydroxy-2-oxo-chromen-4-yl)phenyl]tetradecanamide was prepared from ethyl 3-[2-chloro-4-(tetradecanoylamino)phenyl]-3-oxo-propanoate, following Step 2 of preparing Compound [VII], described below. This resulted in N-[3-chloro-4-(7-hydroxy-2-oxo-chromen-4-yl)phenyl]tetradecanamide as an yellow solid, (0.30 g). LC-MS 498.2 [M+1]⁺, RT 6.99 min.

In Step 6, the compound ethyl (2R)-2-[4-[2-chloro-4-(tetradecanoylamino)phenyl]-2-oxo-chromen-7-yl]oxypropanoate was prepared from N-[3-chloro-4-(7-hydroxy-2-oxo-chromen-4-yl)phenyl]tetradecanamide, following Step 4 of preparing Compound [VII], described below. This resulted in ethyl (2R)-2-[4-[2-chloro-4-(tetradecanoylamino)phenyl]-2-oxo-chromen-7-yl]oxypropanoate as an yellow solid, (0.25 g). LC-MS 598.3 [M+1]⁺, RT 7.83 min.

In Step 7, (2R)-2-[4-[2-Chloro-4-(tetradecanoylamino)ph-enyl]-2-oxo-chromen-7-yl]oxypropanoic acid, Compound [VI], was prepared from ethyl (2R)-2-[4-[2-chloro-4-(tetradecanoylamino)phenyl]-2-oxo-chromen-7-yl]oxypropanoate following the method of Step 6 for preparing Compound [VII], described below. This resulted in a light brown solid (2R)-2-[4-[2-chloro-4-(tetradecanoylamino)phenyl]-2-oxo-chromen-7-yl]oxypropanoic acid, (0.15 g). LC-MS 570.4 [M+1]⁺, RT 2.65 min.

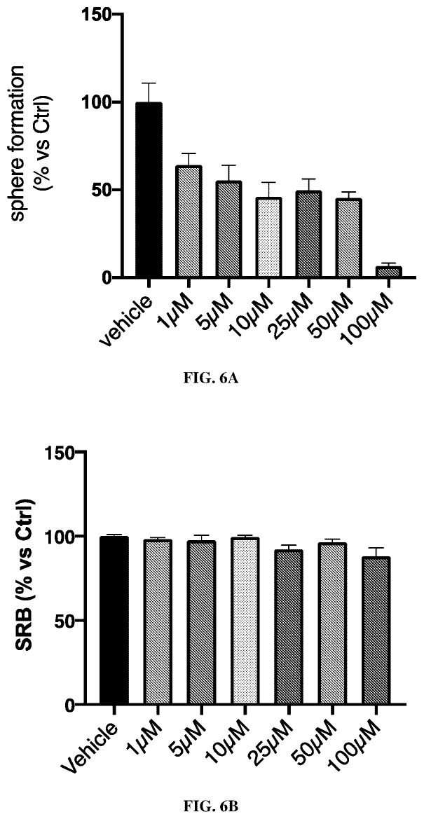

FIGS. 6A and 6B show the mammosphere formation results and SRB assay results on MCF-7 cells, for Compound [VI]. The inhibitory activity can be seen at all tested concentrations, from 1 μM to 100 μM, and over 50% inhibition was achieved at 10 μM. A significant improvement is seen at the increase from 50 μM to 100 μM, and further evaluations between these concentrations are envisioned. With respect to the SRB assay, Compound [VI] showed little toxicity across the tested concentrations, as seen in FIG. 6B.

In some embodiments, the therapeutic agent may take the form of Compound [VII], having the chemical structure shown below:

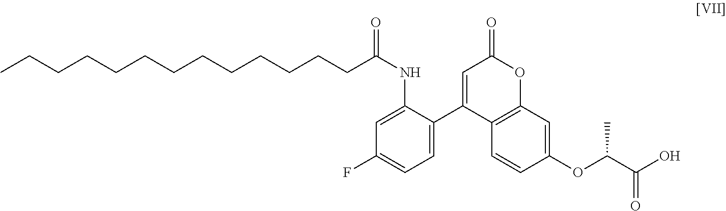 [SMILES: CCCCCCCCCCCCCC(=O)Nc1cc(F)ccc1-c1cc(=O)oc2cc(O[C@H](C)C(=O)O)ccc12]

Compound [VII], (2R)-2-[4-[4-Fluoro-2-(tetradecanoylamino)phenyl]-2-oxo-chromen-7-yl]oxypropanoic acid, was synthesized according to the following approach. In Step 1, ethyl 3-(4-fluoro-2-nitro-phenyl)-3-oxo-propanoate was prepared. To a stirred suspension of ethyl potassium malonate (1.9 g, 11.19 mmol) in dry MeCN (30 ml) under nitrogen atmosphere at +10° C. TEA (1.65 ml, 11.74 mmol) and anhydrous magnesium chloride (1.28 g, 13.42 mmol) was added, and the mixture was stirred at room temperature for 3.5 hours. A solution of 4-fluoro-2-nitrobenzoic acid chloride, prepared according to Archiv der Pharmazie, 1985, vol. 318, No. 1, p. 78-84, (1.14 g, 5.59 mmol) in dry MeCN (2 ml) was added and the mixture was stirred at room temperature for 16 hours. The reaction mixture was poured into a stirred mixture of saturated NH4Cl (2 ml) and EtOAc (50 ml) and the solvent was evaporated under reduced pressure. The solid residue was stirred with 4M HCl (30 ml) at room temperature for 30 minutes and the product was extracted with EtOAc (50 ml). The extract was washed with 4m HCl (30 ml), water (30 ml), brine (30 ml), dried over MgSO4, filtered and the filtrate was concentrated under reduced pressure to yield a crude product. Purification on silica gel (15% EtOAc in iso-hexane) afforded ethyl 3-(4-fluoro-2-nitro-phenyl)-3-oxo-propanoate, (1.16 g, 81%). LC-MS 256.1 [M+1]+, RT 4.08 min.

In Step 2, 4-(4-Fluoro-2-nitro-phenyl)-7-hydroxy-chromen-2-one was produced as an intermediary. To a stirred solution of ethyl 3-(4-fluoro-2-nitro-phenyl)-3-oxo-propanoate (0.60 g, 2.30 mmol) in methane sulphonic acid (4 ml) at room temperature under nitrogen atmosphere resorcinol (0.31 g, 2.20 mmol) was added and the mixture was stirred at +45° C. for 1 hour. The reaction mixture cooled to room temperature, poured into a mixture of water:EtOAc (40 ml:10 ml) and the product was extracted with EtOAc (50 ml). The extract was washed with water (40 ml), brine (40 ml), dried over MgSO4, the solid residue was removed by filtration and the solvent was evaporated under reduced pressure to yield a crude product. Purification on silica gel (40-50% EtOAc in iso-hexane) afforded 4-(4-fluoro-2-nitro-phenyl)-7-hydroxy-chromen-2-one, (0.43 g, 65%). LC-MS 302.1 [M+1]+, RT 4.22 min.

In Step 3, 4-(2-Amino-4-fluoro-phenyl)-7-hydroxy-chromen-2-one was produced. To a stirred ice cold solution of 4-(4-fluoro-2-nitro-phenyl)-7-hydroxy-chromen-2-one (0.21 g, 0.70 mmol) and DiPEA (0.60 ml, 3.50 mmol) in dry DCM (5 ml) under nitrogen atmosphere a solution of trichlorosilane (0.35 ml, 3.5 mmol) in dry DCM (2 ml) was added over 10 minutes. The mixture was stirred at +0° C. for 1 hour, quenched with saturated NaHCO₃ (3 ml), diluted with water (20 ml) and EtOAc (30 ml) to yield a suspension that was filtered through a Celite pad. Solid NaHCO₃ was added to adjust pH to 8-9, the organic phase was separated, washed with brine (20 ml), dried over MgSO4, filtered and the solvent was evaporated under reduced pressure to yield a crude product. Purification on silica gel (40-50% EtOAc in iso-hexane) afforded 4-(2-amino-4-fluoro-phenyl)-7-hydroxy-chromen-2-one, (0.20 g, 99%). LC-MS 302.1 [M+1]+, RT 4.22 min.

In Step 4, ethyl (2R)-2-[4-(2-amino-4-fluoro-phenyl)-2-oxo-chromen-7-yl]oxypropanoate was produced as an intermediary. To a stirred ice cold solution of 4-(2-amino-4-fluoro-phenyl)-7-hydroxy-chromen-2-one (0.33 g, 1.24 mmol), ethyl-S-lactate (212 μl. 1.86 mmol), and triphenylphosphine (0.32 ml, 1.24 mmol) in dry THF (10 ml) under nitrogen atmosphere DEAD (293 μl, 1.86 mmol) was added. The mixture was stirred at +0° C. for 0.5 hour, allowed to warm to room temperature and stirred for 16 hours. The solvent was evaporated under reduced pressure to yield a crude product. Purification on silica gel (50% diethyl ether in iso-hexane) afforded ethyl (2R)-2-[4-(2-amino-4-fluoro-phenyl)-2-oxo-chromen-7-yl]oxypropanoate, (0.43 g, 93%). LC-MS 372.3 [M+1]⁺, RT 5.12 min.

In Step 5, ethyl (2R)-2-[4-[4-fluoro-2-(tetradecanoylamino)phenyl]-2-oxo-chromen-7-yl]oxypropanoate was produced. To a stirred solution of ethyl (2R)-2-[4-(2-amino-4-fluoro-phenyl)-2-oxo-chromen-7-yl]oxypropanoate ((0.26 g, 0.70 mmol) in dry pyridine (3 ml) at room temperature under nitrogen atmosphere tetradecanoyl chloride (380 μl, 1.40 mmol) was added in two portions. The mixture was stirred for 16 hours and the solvent was evaporated under reduced pressure to yield a crude product. The residue was dissolved in iso-propanol (10 ml), diluted with EtOAc (20 ml), washed with saturated NaHCO₃ (20 ml), brine (20 ml), dried over MgSO₄, filtered and the solvent was evaporated under reduced pressure to yield a crude product. Purification on silica gel (10-15% EtOAc in iso-hexane) afforded ethyl (2R)-2-[4-[4-fluoro-2-(tetradecanoylamino)phenyl]-2-oxo-chromen-7-yl]oxypropanoate, (0.21 g, 51%). LC-MS 580.4 [M+1]⁺, RT 7.20 min.

In Step 6, (2R)-2-[4-[4-Fluoro-2-(tetradecanoylamino)phenyl]-2-oxo-chromen-7-yl]oxypropanoic acid, Compound [VII], was prepared. To a stirred solution of ethyl (2R)-2-[4-[4-fluoro-2-(tetradecanoylamino)phenyl]-2-oxo-chromen-7-yl]oxypropanoate ((0.20 g, 0.34 mmol) in THF (4 ml) at room temperature under nitrogen atmosphere 1M NaOH (2 ml, 2.00 mmol) was added. The mixture was stirred for 5 hours, diluted with EtOAc (30 ml) washed with 2H HCl (10 ml), water (10 ml), brine (20 ml), dried over MgSO₄, filtered and the solvent was evaporated under reduced pressure to yield a crude product. Purification on silica gel (4% MeOH in DCM) afforded (2R)-2-[4-[4-fluoro-2-(tetradecanoylamino)phenyl]-2-oxo-chromen-7-yl]oxypropanoic acid, (0.15 g, 79%). LC-MS 554.5 [M+1]⁺, RT 7.00 min.

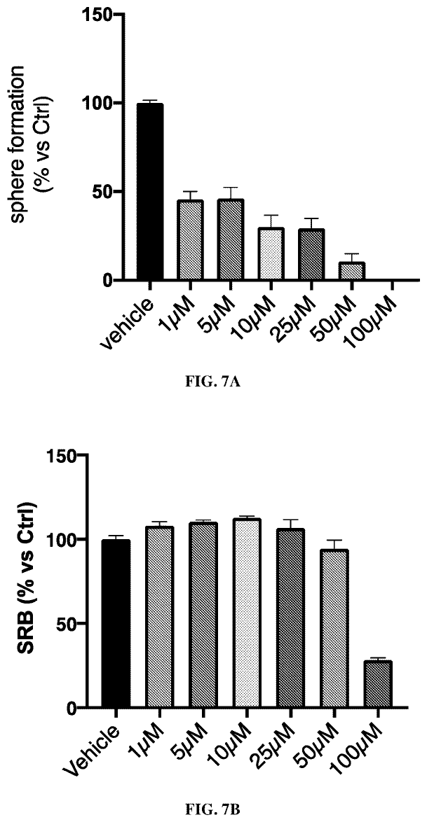

FIGS. 7A and 7B show the mammosphere formation results and SRB assay results on MCF-7 cells, for Compound [VII]. The inhibitory activity can be seen at all tested concentrations, from 1 μM to 100 μM, and over 50% inhibition was achieved at 1 μM. At a concentration of 100 μM, Compound [VII] exhibited a 100% inhibition of MCF-7 cells. With respect to the SRB assay, FIG. 7B shows that Compound [VII] has little toxicity across the tested concentrations below 100 μM.

The following example describes the synthesis of iso-Butyl N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]carbamate (shown below).

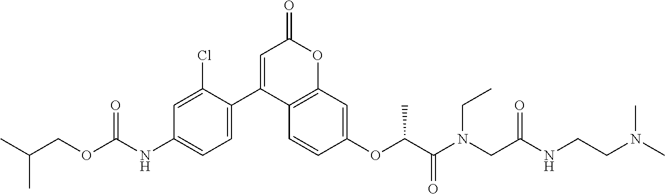 [SMILES: CCN(CC(=O)NCCN(C)C)C(=O)[C@@H](C)Oc1ccc2c(-c3ccc(NC(=O)OCC(C)C)cc3Cl)cc(=O)oc2c1]

In Step 1, (2R)-2-[4-(4-Amino-2-chloro-phenyl)-2-oxo-chromen-7-yl]oxy-N-[2-[2-(dimethylamino)ethylamino]-2-oxo-ethyl]-N-ethyl-propanamide. To a stirred solution of tert-butyl N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]carbamate (0.125 g, 0.20 mmol) in dry DCM (3 ml) at room temperature under nitrogen atmosphere, TFA (3 ml) was added. The mixture was stirred for 1 hour and the solvent was evaporated under reduced pressure to yield a crude product. The residue was dissolved in EtOAc (30 ml), saturated NaHCO₃ (10 ml), brine (10 ml), dried over MgSO₄, filtered and the solvent was evaporated under reduced pressure to yield (2R)-2-[4-(4-amino-2-chloro-phenyl)-2-oxo-chromen-7-yl]oxy-N-[2-[2-(dimethylamino)ethylamino]-2-oxo-ethyl]-N-ethyl-propanamide, (0.11 g). LC-MS 515.0 [M+1]⁺, RT 3.31 min.

In Step 2, iso-Butyl N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]carbamate was prepared from (2R)-2-[4-(4-amino-2-chloro-phenyl)-2-oxo-chromen-7-yl]oxy-N-[2-[2-(dimethylamino)ethylamino]-2-oxo-ethyl]-N-ethyl-propanamide the following the method in Example 6 Step 3 in absence of acid affording tert-butyl N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]carbamate as a light brown solid, (10.1 mg). LC-MS 615.2 [M+1]⁺, RT 4.38 min.

The following demonstrates the synthesis of N-[3-Chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]dodecanamide (shown below).

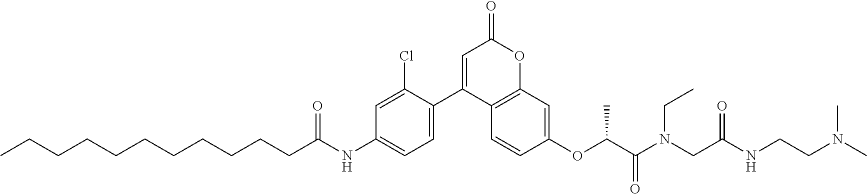 [SMILES: CCCCCCCCCCCC(=O)Nc1ccc(-c2cc(=O)oc3cc(O[C@H](C)C(=O)N(CC)CC(=O)NCCN(C)C)ccc23)c(Cl)c1]

To a stirred solution of (2R)-2-[4-(4-amino-2-chloro-phenyl)-2-oxo-chromen-7-yl]oxy-N-[2-[2-(dimethylamino)ethylamino]-2-oxo-ethyl]-N-ethyl-propanamide (30 mg, 0.058 mmol) and pyridine (0.5 ml) in methyltetrahydrofuran (2 ml) at room temperature under nitrogen atmosphere lauryl chloride (16 μl, 0.073 mmol) was added. The mixture was stirred for 16 hours and the solvent was evaporated under reduced pressure. The residue was dissolved in EtOAc (30 ml), water (10 ml), brine (10 ml), dried over MgSO₄, filtered and the solvent was evaporated under reduced pressure to yield the residue was dissolved in EtOAc (20 ml), washed with water (10 ml), brine (10 ml), dried over MgSO₄, filtered and the filtrate was evaporated under reduced pressure to yield a crude product. Purification on silica gel (5-10% MeOH in DCM with 1-2% 7M NH₃ in MeOH) afforded N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]dodecanamide, (27.3 mg). LC-MS 697.2 [M+1]⁺, RT 5.47 min.

The next example describes the synthesis of N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]hexanamide (shown below).

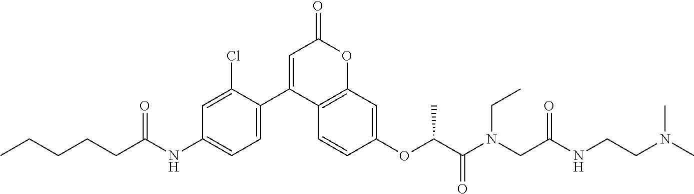 [SMILES: CCCCCC(=O)Nc1ccc(-c2cc(=O)oc3cc(O[C@H](C)C(=O)N(CC)CC(=O)NCCN(C)C)ccc23)c(Cl)c1]

The title compound was prepared from (2R)-2-[4-(4-amino-2-chloro-phenyl)-2-oxo-chromen-7-yl]oxy-N-[2-[2-(dimethylamino)ethylamino]-2-oxo-ethyl]-N-ethyl-propanamide the following the method in Example 9 affording N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]hexanamide as a light brown solid, (20.0 mg). LC-MS 613.1 [M+1]⁺, RT 4.93 min.

It should be appreciated that the lactic acid moiety of the propanamido portion of Formula 1 may be modified with certain side chains—or the methyl replaced with hydrogen—in some embodiments. For example, some embodiments of compounds under the present approach have the generic chemical structure of Formula 2, shown below:

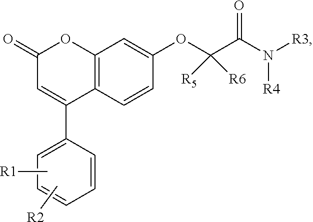

or pharmaceutically acceptable salts thereof, in which R1, R2, R3, and R4 are as described above with respect to Formula 1, and R5 and R6 may be the same or different, and may be hydrogen, substituted or unsubstituted C1-C10 alkyl, substituted or unsubstituted C3-C8-cycloalkyl, substituted or unsubstituted pyridine, substituted or unsubstituted C2-C10 carboxyl, substituted or unsubstituted C2-C10 alkene, substituted or unsubstituted C2-C10 alkyne, substituted or unsubstituted C2-C10 ketone, substituted or unsubstituted C2-C10 aldehyde, substituted or unsubstituted C2-C10 ether, substituted or unsubstituted C2-C10 ester, substituted or unsubstituted C2-C10 amine, substituted or unsubstituted C2-C10 amide, substituted or unsubstituted C2-C10 alkyl-amide, substituted or unsubstituted phenol, or benzoic acid.

Preferably, R5 and R6 are the same or different, and are H or a substituent in substituted alpha-hydroxy acid (AHH). Below are examples of structures according to the present approach, demonstrating variations in R5 and R6, and including various enantiomers thereof motifs. It should be appreciated that only a portion of the structure is illustrated, and that the entirety of the structure for a given example may be understood in view of Formula 2 above.

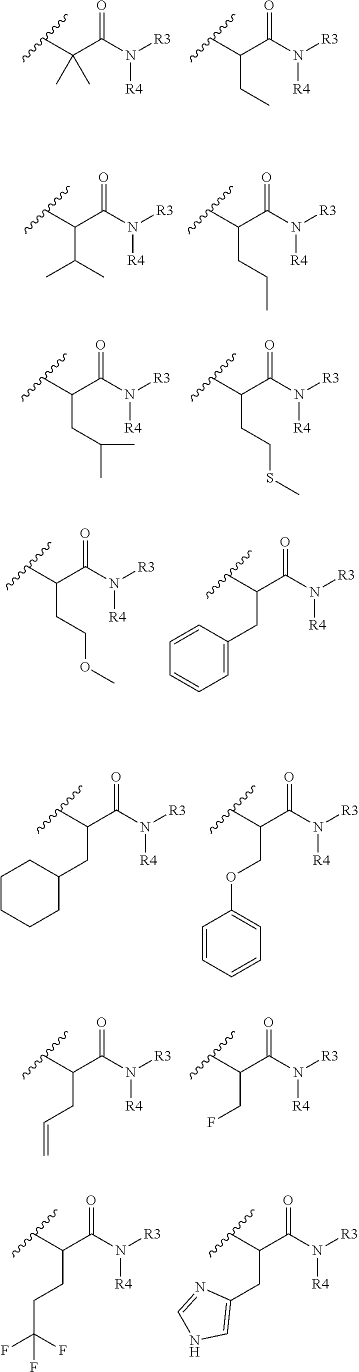

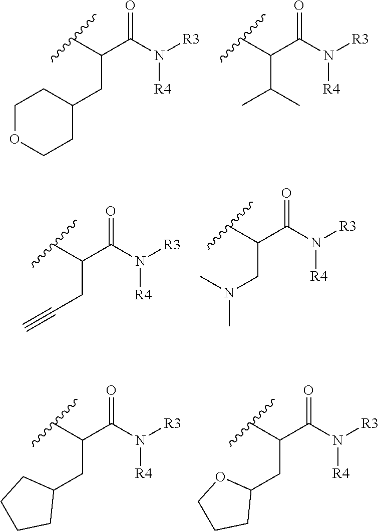

Synthesis of compounds according to Formula 2 may be made generally following the general synthesis example illustrated above. Below is an example of a general scheme for synthesizing compounds according to Formula 2.

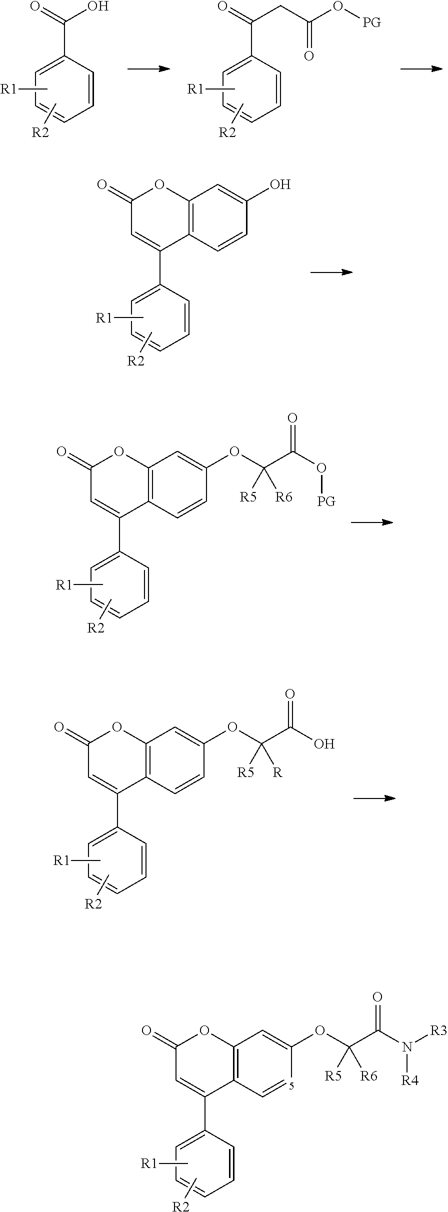

where R1-R6 are as described above. In one demonstrative embodiment, a compound may have the structure of Compound [VIII], below, in which R1 is 14-carbon amide group, R2 is chloro, R3 and R4 are methyl, and R5 and R6 are hydrogen:

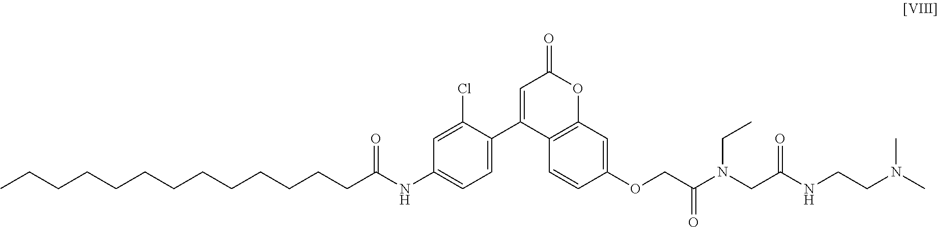 [SMILES: CCCCCCCCCCCCCC(=O)Nc1ccc(-c2cc(=O)oc3cc(OCC(=O)N(CC)CC(=O)NCCN(C)C)ccc23)c(Cl)c1]

The IUPAC name for Compound [VIII] is N-[3-chloro-4-[7-[2-[[2-[2-(dimethylamino)ethylamino]-2-oxo-ethyl]-ethyl-amino]-2-oxo-ethoxy]-2-oxo-chromen-4-yl]phenyl]tetradecanamide. A demonstrative synthesis scheme for Compound [VII](and similar embodiments of varying alkyl chain length) is shown below.

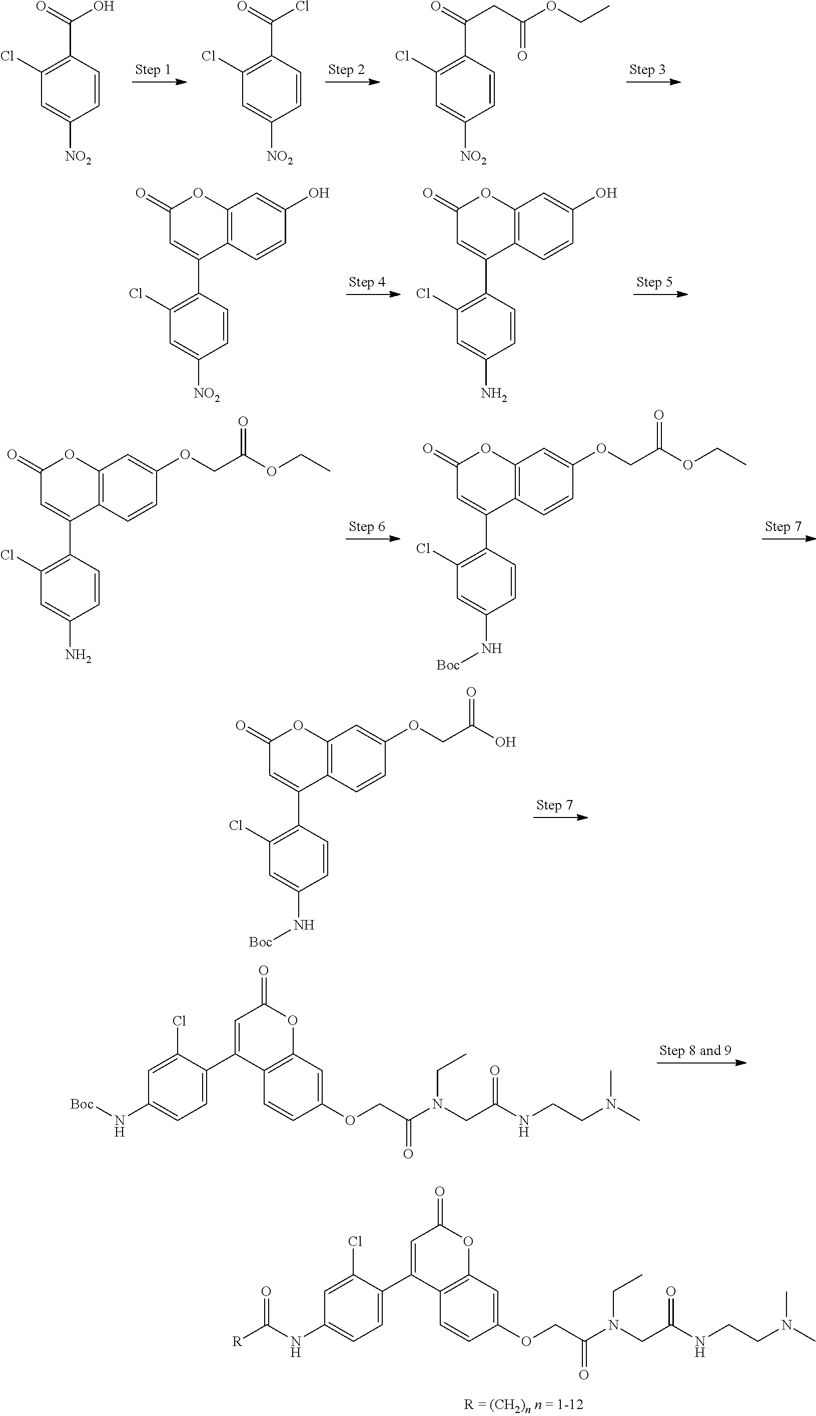

In the foregoing scheme, it should be appreciated that the person having an ordinary level of skill in the art may identify more than one approach to perform one or more of the numbered steps. As an example, Compound [VIII] can be synthesized from intermediate N-[3-chloro-4-(7-hydroxy-2-oxo-chromen-4-yl)phenyl]tetradecanamide, using the following scheme:

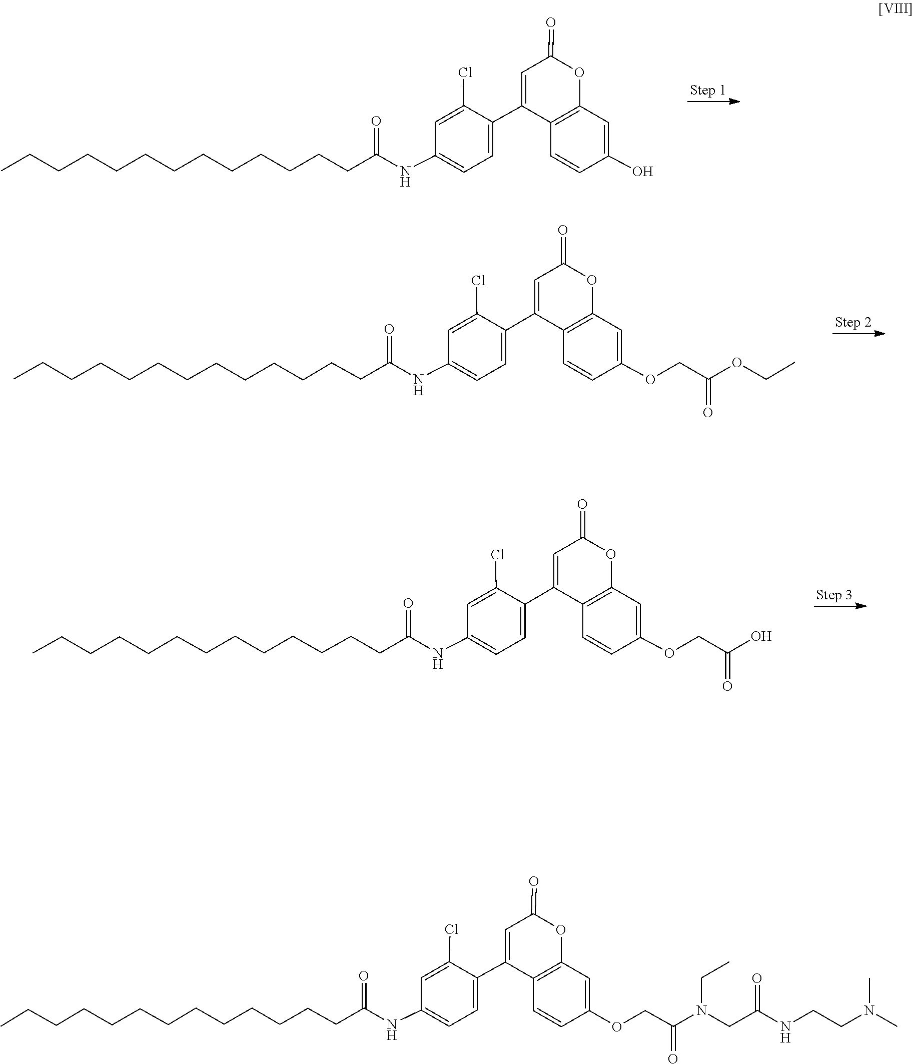

In Step 1, the compound ethyl ethyl 2-[4-[2-chloro-4-(tetradecanoylamino)phenyl]-2-oxo-chromen-7-yl]oxyacetate was prepared from N-[3-chloro-4-(7-hydroxy-2-oxo-chromen-4-yl)phenyl]tetradecanamide, following Step 4 of preparing Compound [VII], described above. This resulted in ethyl 2-[4-[2-chloro-4-(tetradecanoylamino)phenyl]-2-oxo-chromen-7-yl]oxyacetate as a light brown solid, (0.25 g). LC-MS 584.3 [M+1]⁺, RT 6.95 min.

In Step 2, 2-[4-[2-chloro-4-(tetradecanoylamino)phenyl]-2-oxo-chromen-7-yl]oxyacetic acid, was prepared from ethyl 2-[4-[2-chloro-4-(tetradecanoylamino)phenyl]-2-oxo-chromen-7-yl]oxyacetate following the method of Step 6 for preparing Compound [VII], described above. This resulted in a colorless solid 2-[4-[2-chloro-4-(tetradecanoylamino)phenyl]-2-oxo-chromen-7-yl]oxyacetic acid, (0.05 g). LC-MS 556.1 [M+1]⁺, RT 6.64 min.

In step 3, N-[3-chloro-4-[7-[2-[[2-[2-(dimethylamino)ethylamino]-2-oxo-ethyl]-ethyl-amino]-2-oxo-ethoxy]-2-oxo-chromen-4-yl]phenyl]tetradecanamide [VIII] was synthesized with following method: To a stirred solution of 2-[4-[2-chloro-4-(tetradecanoylamino)phenyl]-2-oxo-chromen-7-yl]oxyacetic acid (50 mg, 0.09 mmol) in dry DCM (3 ml) at room temperature under nitrogen atmosphere NMM (100 μl, 0.72 mmol) and EDC hydrochloride (32 mg, 0.17 mmol), HOBt hydrate (24 mg, 0.17 mmol) and a solution of N-[2-(dimethylamino)ethyl]-2-(ethylamino)acetamide trifluoroacetate (240 mg, 0.60 mmol) in dry DMF (4 ml) was added. The mixture was stirred for 16 hours and the solvent was evaporated under reduced pressure to yield a crude product. Purification on silica gel (5-13% MeOH in DCM with 1-2% 7M NH3 in MeOH) afforded N-[3-chloro-4-[7-[2-[[2-[2-(dimethylamino)ethylamino]-2-oxo-ethyl]-ethyl-amino]-2-oxo-ethoxy]-2-oxo-chromen-4-yl]phenyl]tetradecanamide, (50 mg, 78%). LC-MS 711.5 [M+1]⁺, RT 5.89 min.

Another demonstrative example of an embodiment is Compound [IX], shown below. Compound [IX] has an IUPAC name of N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]tetradecanamide.

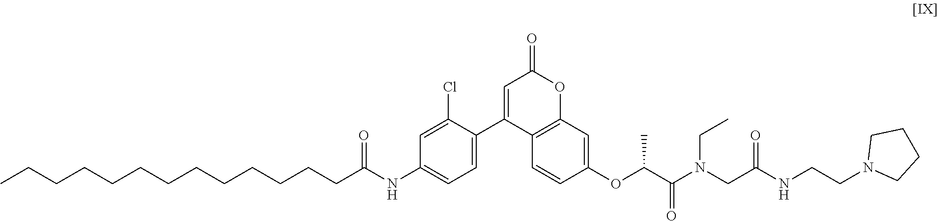 [SMILES: CCCCCCCCCCCCCC(=O)Nc1ccc(-c2cc(=O)oc3cc(O[C@H](C)C(=O)N(CC)CC(=O)NCCN4CCCC4)ccc23)c(Cl)c1]

Compound [IX] was synthesized as follows. In Step 1, to a stirred solution of (2R)-2-[4-[2-chloro-4-(tetradecanoylamino)phenyl]-2-oxo-chromen-7-yl]oxypropanoic acid (see Compound [VI], described above] (0.35 g, 0.61 mmol) and N-ethylglycine ethyl ester trifluoroacetate (0.22 g, 1.22 mmol), in a mixture of DCM (10 ml) and DMF (5 ml) at room temperature under nitrogen atmosphere N-(3-dimethylamoinopropyl)-N′-ethylcarbodiimide hydrochloride (0.23 g, 1.22 mmol), 1-hydroxybenzotriazole hydrate (0.17 g, 1.22 mmol) and NMM (0.33 ml, 3.0 mmol) was added. The mixture was stirred for 16 hours, solvent was evaporated under reduced pressure to yield a crude product. Purification on silica gel (50-100% EtOAc in iso-hexane afforded ethyl 2-[[(2R)-2-[4-[2-chloro-4-(tetradecanoylamino)phenyl]-2-oxo-chromen-7-yl]oxypropanoyl]-ethyl-amino]acetate (0.28 g). LC-MS 683.3 [M+1]⁺, RT 6.89 min.

In Step 2, 2-[[(2R)-2-[4-[2-chloro-4-(tetradecanoylamino)phenyl]-2-oxo-chromen-7-yl]oxypropanoyl]-ethyl-amino]acetic acid was prepared from ethyl 2-[[(2R)-2-[4-[2-chloro-4-(tetradecanoylamino)phenyl]-2-oxo-chromen-7-yl]oxypropanoyl]-ethyl-amino]acetate following the method in Step 6 of Compound [IV], affording 2-[[(2R)-2-[4-[2-chloro-4-(tetradecanoylamino)phenyl]-2-oxo-chromen-7-yl]oxypropanoyl]-ethyl-amino]acetic acid as a light brown solid, (0.16 g). LC-MS 655.3 [M+1]⁺, RT 6.63 min.

In Step 3, N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]tetradecanamide (Compound [XI]) was prepared from 2-[[(2R)-2-[4-[2-chloro-4-(tetradecanoylamino)phenyl]-2-oxo-chromen-7-yl]oxypropanoyl]-ethyl-amino]acetic acid and 1-(2-aminoethyl)pyrrolidine following the method in Step 1 affording N-[3-chloro-4-[7-[(1R)-2-[[2-[2-(dimethylamino)ethylamino]-2-oxo-ethyl]-ethyl-amino]-1-methyl-2-oxo-ethoxy]-2-oxo-chromen-4-yl]phenyl]tetradecanamide as a white solid, (15.9 mg). LC-MS 751.3 [M+1]⁺, RT 6.06 min.

Another demonstrative example of an embodiment is Compound [X], shown below. Compound [X] has an IUPAC name of N-[3-chloro-4-[7-[(1R)-2-[ethyl-[2-[(1-methylpyrrolidin-3-yl)amino]-2-oxo-ethyl]amino]-1-methyl-2-oxo-ethoxy]-2-oxo-chromen-4-yl]phenyl]tetradecanamide.

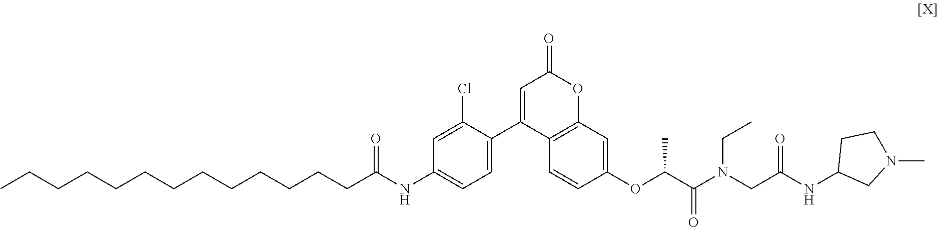 [SMILES: CCCCCCCCCCCCCC(=O)Nc1ccc(-c2cc(=O)oc3cc(O[C@H](C)C(=O)N(CC)CC(=O)NC4CCN(C)C4)ccc23)c(Cl)c1]

Compound [X] was prepared from (2R)-2-[4-[2-chloro-4-(tetradecanoylamino)phenyl]-2-oxo-chromen-7-yl]oxypropanoic acid (Compound [VI]) and 1-methylpyrrolidin-3-amine following the method in Step 1 of Compound [IX], affording N-[3-chloro-4-[7-[(1R)-2-[ethyl-[2-[(1-methylpyrrolidin-3-yl)amino]-2-oxo-ethyl]amino]-1-methyl-2-oxo-ethoxy]-2-oxo-chromen-4-yl]phenyl]tetradecanamide as a white solid, (16.4 mg). LC-MS 737.4 [M+1]⁺, RT 5.97 min.

Another demonstrative example of an embodiment is Compound [XI], shown below. Compound [XI] has an IUPAC name of N-[3-chloro-4-[7-[(1R)-2-[ethyl-[2-(isopentylamino)-2-oxo-ethyl]amino]-1-methyl-2-oxo-ethoxy]-2-oxo-chromen-4-yl]phenyl]tetradecanamide.

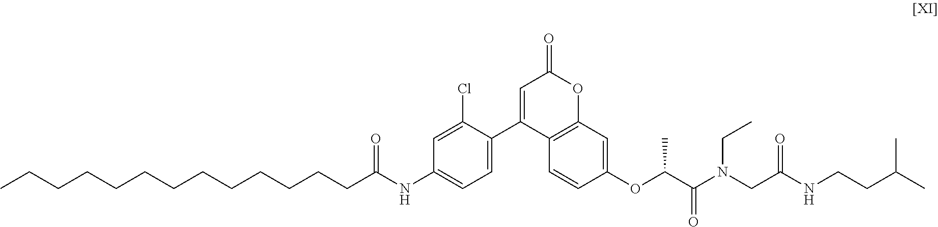 [SMILES: CCCCCCCCCCCCCC(=O)Nc1ccc(-c2cc(=O)oc3cc(O[C@H](C)C(=O)N(CC)CC(=O)NCCC(C)C)ccc23)c(Cl)c1]

Compound [XI] was prepared from (2R)-2-[4-[2-chloro-4-(tetradecanoylamino)phenyl]-2-oxo-chromen-7-yl]oxypropanoic acid (Compound [VI]) and 3-methylbutylamine following the method in Step 1 of Compound [IX] affording N-[3-chloro-4-[7-[(1R)-2-[ethyl-[2-(isopentylamino)-2-oxo-ethyl]amino]-1-methyl-2-oxo-ethoxy]-2-oxo-chromen-4-yl]phenyl]tetradecanamide as a white solid, (24.9 mg). LC-MS 724.4 [M+1]⁺, RT 6.78 min

The description of synthesis methods and reaction schemes use the following abbreviations: acetonitrile (MeCN), ethyl acetate (EtOAc), methanol (MeOH), hydrochloric acid (HCl), magnesium sulphate (MgSO₄), di-isopropyl ethyl amine (DiPEA), dichloromethane (DCM), sodium hydrogen carbonate NaHCO₃, isopropanol (IPA), tetrahydrofuran (THF), ammonium chloride (NH₄Cl), sodium hydroxide (NaOH), N-(3-Dimethylaminopropyl)-N′-ethylcarbodiimide hydrochloride (EDC HCl), triethylamine (TEA), N-methylmorpholine (NMM), diethylazodicarboxylate (DEAD), dimethylacetamide (DMA), dimethylformamide (DMF), ammonia (NH₃), and 4-dimethylaminopyridine (DMAP), trifluoroacetic acid (TFA), 1-hydroxybenzotriazole (HOBt), ammonia (NH₃).

The liquid chromatography-mass spectroscopy data was prepared using a Waters Sunfire C18 30×4.6 mm column. Gradient eluent: 20-100% acetonitrile/water containing 0.05% formic acid. Time: 0-10 min.

Further evaluation of both efficacy and toxicity was performed on human breast tumors initiated from the MDA-MB-231 cell line in chicken embryos using the chick chorioallantoic membrane assay (“CAM Assay”) available from Inovotion (Cambridge, Massachusetts). Compounds of the present approach have showed significant effects on MDA-MB-231 cancer cell metastasis, without toxicity towards embryos in vivo. An inoculum of 1×10⁶ MDA-MB-231 cells was added onto the CAM of each egg (day E9) and then eggs were randomized into groups. On day E10, tumors were detectable and they were then treated daily for 8 days with vehicle alone (1% DMSO in PBS), or a compound of the present approach. After 8 days of drug administration, on day E18 all tumors were weighed, and the lower CAM was collected to evaluate the number of metastatic cells, as analyzed by qPCR with specific primers for Human Alu sequences.

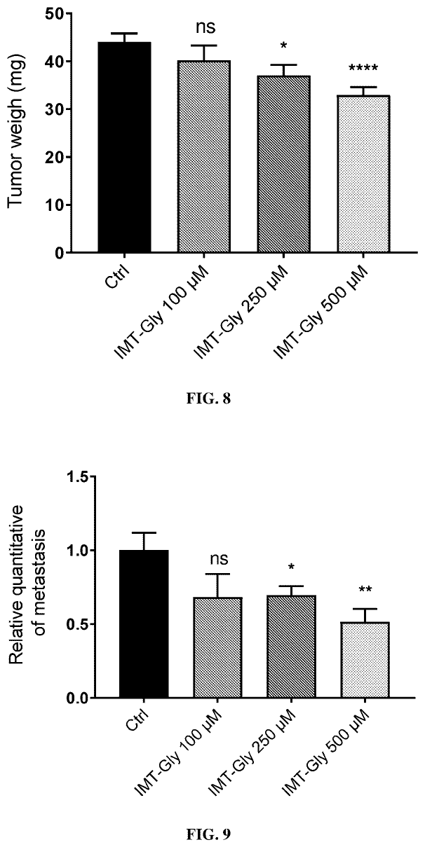

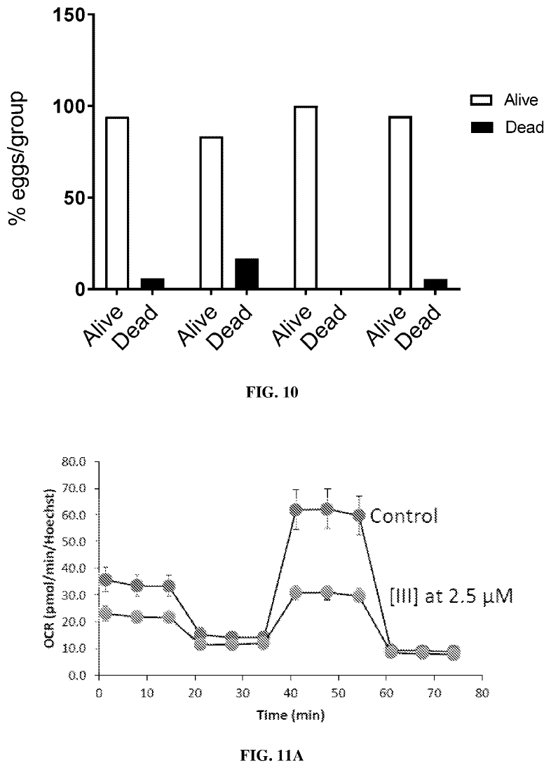

FIG. 8 shows a quantitative evaluation of tumor growth using the CAM assay, for different concentrations of an embodiment of the present approach, Compound [III]. The results show a dose-dependent reduction in tumor growth for cells, illustrating the pharmaceutical activity of the demonstrative compound. Similar activity is present in the other compounds disclosed herein. FIG. 9 shows a quantitative evaluation of metastatic invasion using the CAM assay, for different concentrations of an embodiment of the present approach, Compound [III]. A dose-dependent reduction in metastasis is evident for the demonstrative compound. Other compounds described herein have similar activity. For FIGS. 8 and 9, the data was analyzed via t-test, with a P>0.05. A single * indicates P<0.05, ** indicates P<0.001, and **** indicates P>0.0001. FIG. 10 shows a toxicity assessment using the CAM assay, for different concentrations of Compound [III]. As can be seen, the demonstrative compound is non-toxic towards normal cells. The selectivity towards metabolically active cancer stem cells is a common property of compounds according to the present approach. Table 1, below, summarizes the toxicity analysis from the CAM assay.

TABLE 1
Chick Embryo Toxicity of Compound [III].

| Group | Group | | | | | |
|---|---|---|---|---|---|---|
| # | Description | Total | Alive | Dead | % Alive | % Dead |
| 1 | Neg. Ctrl. | 17 | 16 | 1 | 94.12 | 5.88 |
| 2 | [III] - 100 μM | 18 | 15 | 3 | 83.33 | 16.67 |
| 3 | [III] - 250 μM | 18 | 18 | 0 | 100 | 0 |
| 4 | [III] 500 μM | 18 | 17 | 1 | 94.44 | 5.56 |

This demonstrates that the compounds of the present approach are effective at preventing or reducing the likelihood of metastasis. It should be appreciated that the same CAM assay may be used to reproduce these properties in the embodiments of the present approach. Additionally, little-to-no embryo toxicity was observed for the demonstrative compound using the CAM assay. The disclosed compounds have efficacy as an anti-metastatic agent, selectively inhibiting tumor metastasis, without significant toxicity or antibiotic activity.

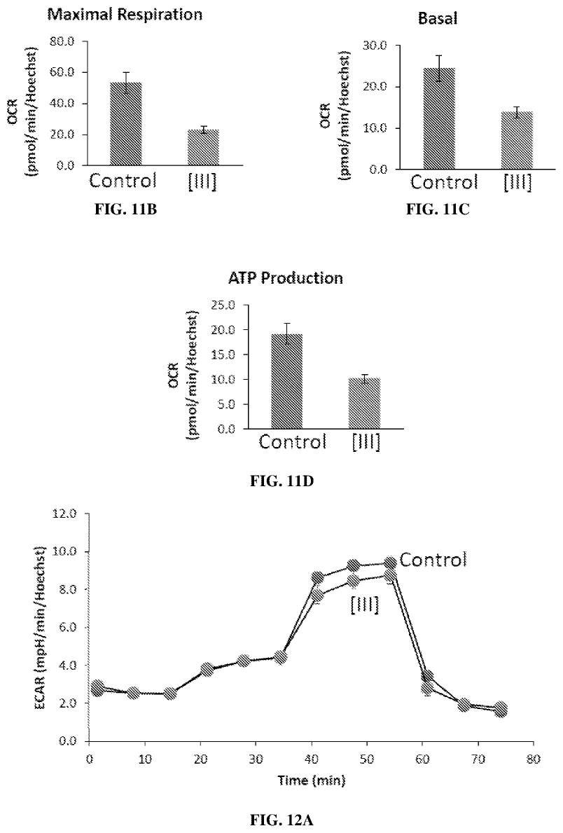

To examine the effects of the compounds described herein on mitochondrial respiration and aerobic glycolysis, adherent MCF7 cells were treated with a demonstrative compound, and then OCR and ECAR were measured. FIGS. 11A-D show mitochondrial respiration, basal respiration, maximal respiration, and ATP production results for metabolic flux analysis of adherent MCF7 cells were treated with Compound [III] at 2.5 μM. After treatment, the mitochondrial oxygen consumption rate (OCR) was measured using the Seahorse XFe96 analyzer. Data is expressed as percentage of OCR versus control.

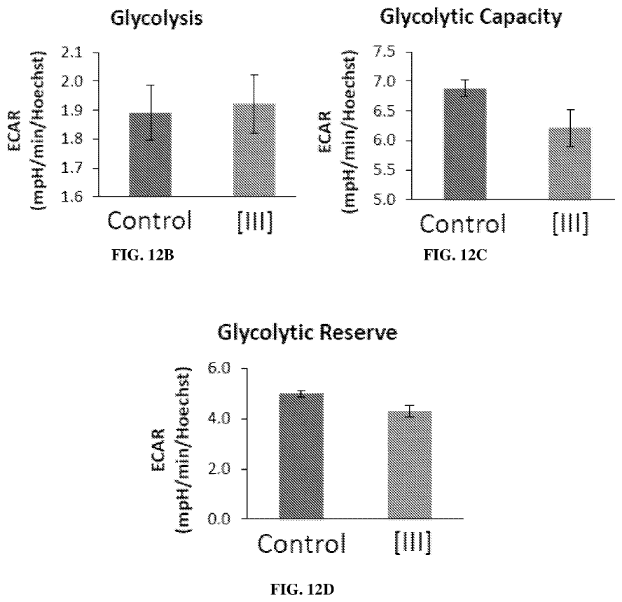

FIGS. 12A-D show the results of glycolytic function analysis for treatment with Compound [III] at 2.5 μM. Glycolytic function, basal glycolysis, induced glycolysis, and compensatory glycolysis, respectively. The data show that Compound [III] significantly inhibited basal and maximal OCR, as well as ATP production levels, as compared to vehicle-alone control cells. In contrast, ECAR levels were largely comparable between the control and the demonstrative compound. These results are consistent with preliminary evaluations of other compounds described herein, although evaluation of different compounds and concentrations for compounds is ongoing. All data was normalized for cell number. Statistical analysis was conducted using one-way ANOVA.

The data directly validates how Compound [III] specifically functions as a mitochondrial inhibitor of respiration and ATP production, with little or no effects on glycolysis. Further, the results are consistent with the understanding that the compounds of the present approach, such as Compound [II], are inhibitors of mitochondrial transcription (IMT), by targeting the POLRMT enzyme.

The following paragraphs describe the materials and assays used to generate the data described herein. It should be appreciated that the person having an ordinary level of skill in the art may perform the same assays as described herein, and/or may utilize other assays generally known in the art to assess the physical, chemical, and pharmaceutical properties of a compound as described herein.

Reagents and Model Cell Lines: It should be apparent that other cell lines may be used without departing from the present approach. The human breast adenocarcinoma cell line MCF-7 was from the American Type Culture Collection (ATCC). hTERT-BJ1 cells were from Clontech, Inc. MCF-7 and hTERT-BJ 1 cells were grown in DMEM supplemented with 10% fetal bovine serum, GlutaMAX and 1% penicillin-streptomycin and incubated at 37 C in a humidified 5% C02 incubator. The medium was changed 2-3 times/week.

3D Anchorage Independent Growth Assay: This assay is also referred to as the mammosphere formation assay. A single-cell suspension was prepared using enzymatic, and manual disaggregation (25-g needle). Then, cells were plated at a density of 500 cells/cm2 in mammosphere medium (DMEM-F12+1X B-27 Plus Supplement+20 ng/ml EGF+Pen/Strep) under non-adherent conditions, in culture dishes pre-coated with (2-hydroxyethylmethacrylate) (poly-HEMA, Sigma Aldrich Inc.), called “mammosphere plates.” Cells were grown for 5 days and maintained in a humidified incubator at 37° C. After 5 days of culture, 3D-mammospheres >50 μm were counted using an eye piece (“graticule”), and the percentage of cells plated which formed spheres was calculated and is referred to as percent mammosphere formation, and was normalized to one (1=100% MFE). 3D mammosphere formation efficiency (MFE) was analyzed in both the ATP-low and ATP-high sub-populations of cells. All 3D mammosphere experiments were performed in triplicate, at least 3 times independently.

Statistical Analysis: All analyses were performed with GraphPad Prism 6. Data were represented as mean±SD (or ±SEM where indicated). All experiments were conducted at least 3 times independently, with >3 technical replicates for each experimental condition tested (unless stated otherwise, e.g., when representative data is shown). Statistically significant differences were determined using the Student's t-test or the analysis of variance (ANOVA) test. For the comparison among multiple groups, one-way ANOVA was used to determine statistical significance. p<0.05 was considered significant.

The present approach includes methods of confirming cell viability. Persons of skill in the art may select one or more methods for confirming cell viability suitable for the particular embodiment. The inventors initially used the sulphorhodamine (SRB) assay, which is based on the measurement of cellular protein content. After treatment for 72 hours in 96-well plates, cells were fixed with 10% trichloroacetic acid (TCA) for 1 hour in the cold room, and were dried overnight at room temperature. Then, cells were incubated with SRB for 15 min, washed twice with 1% acetic acid, and air dried for at least 1 hour. Finally, the protein-bound dye was dissolved in a 10 mM Tris, pH 8.8 solution and read using the plate reader at 540-nm. Using the SRB assay, the inventors selected only the compounds depleting ATP levels without prominent cytotoxicity for further analysis. Prominent cytotoxicity was defined as fewer than 30% of cells still on the plate. Of course, embodiments employing other cell viability confirmation methodology may select compounds for further analysis based on other considerations as may be known in the art.

The therapeutic agents may be used in the form of pharmaceutical compositions which may be prepared using one or more known methods. For example, a pharmaceutical composition may be prepared by using diluents or excipients such as, for example, one or more fillers, bulking agents, binders, wetting agents, disintegrating agents, surface active agents, lubricants, and the like as are known in the art. Various types of administration unit forms can be selected depending on the therapeutic purpose(s). Examples of forms for pharmaceutical compositions include, but are not limited to, tablets, pills, powders, liquids, suspensions, emulsions, granules, capsules, suppositories, injection preparations (solutions and suspensions), topical creams, and other forms as may be known in the art. For the purpose of shaping a pharmaceutical composition in the form of tablets, any excipients which are known may be used, for example carriers such as lactose, white sugar, sodium chloride, glucose, urea, starch, calcium carbonate, kaolin, cyclodextrins, crystalline cellulose, silicic acid and the like; binders such as water, ethanol, propanol, simple syrup, glucose solutions, starch solutions, gelatin solutions, carboxymethyl cellulose, shelac, methyl cellulose, potassium phosphate, polyvinylpyrrolidone, etc. Additionally, disintegrating agents such as dried starch, sodium alginate, agar powder, laminalia powder, sodium hydrogen carbonate, calcium carbonate, fatty acid esters of polyoxyethylene sorbitan, sodium laurylsulfate, monoglyceride of stearic acid, starch, lactose, etc., may be used. Disintegration inhibitors such as white sugar, stearin, coconut butter, hydrogenated oils; absorption accelerators such as quaternary ammonium base, sodium laurylsulfate, etc., may be used. Wetting agents such as glycerin, starch, and others known in the art may be used. Adsorbing agents such as, for example, starch, lactose, kaolin, bentonite, colloidal silicic acid, etc., may be used. Lubricants such as purified talc, stearates, boric acid powder, polyethylene glycol, etc., may be used. If tablets are desired, they can be further coated with the usual coating materials to make the tablets as sugar coated tablets, gelatin film coated tablets, tablets coated with enteric coatings, tablets coated with films, double layered tablets, and multi-layered tablets. Pharmaceutical compositions adapted for topical administration may be formulated as ointments, creams, suspensions, lotions, powders, solutions, pastes, gels, foams, sprays, aerosols, or oils. Such pharmaceutical compositions may include conventional additives which include, but are not limited to, preservatives, solvents to assist drug penetration, co-solvents, emollients, propellants, viscosity modifying agents (gelling agents), surfactants, and carriers.

The present approach may be used to prevent and/or reduce the likelihood of tumor recurrence, metastasis. Anti-cancer treatments often fail because the tumor recurs or metastasizes, particularly after surgery. CSC mitochondrial activity is, at least in part, responsible for these causes of treatment failure. Embodiments of the present approach may be used in situations where conventional cancer therapies fail, and/or in conjunction with anti-cancer treatments to prevent or reduce the likelihood of failure due to tumor recurrence and/or metastasis.

The present approach provides for methods of selectively targeting cancer cells. The target cancer cell may be at least one of a CSC, an energetic cancer stem cell (e-CSC), a circulating tumor cell (CTC, a seed cell leading to the subsequent growth of additional tumors in distant organs, a mechanism responsible for a large fraction of cancer-related deaths), and a therapy-resistant cancer cell (TRCC, a cell that has developed a resistance to one or more of chemotherapies, radiotherapies, and other common cancer treatments).

As described in Applicant's co-pending U.S. Provisional Patent Application Nos. 62/686,881, filed Jun. 19, 2018, and 62/731,561, filed Sep. 14, 2018, and incorporated by reference in their entirety, e-CSCs represent a CSC phenotype associated with proliferation. In addition to bulk cancer cells and CSCs, it should be appreciated that the present approach may be used to target a hyper-proliferative cell sub-population that the inventors refer to as e-CSCs, which show progressive increases in sternness markers (ALDH activity and mammosphere-forming activity), highly elevated mitochondrial mass, and increased glycolytic and mitochondrial activity.

In view of the foregoing, it should be appreciated that the present approach may take a wide variety of forms, depending on the embodiment. For example, embodiments of the present approach may take the form of a composition, and in particular a pharmaceutical composition. The therapeutic compound may be the active ingredient, and may be present in a pharmaceutically-effective amount.

Embodiments of the present approach may also take the form of methods for preventing or reducing the likelihood of at least one of tumor recurrence and metastasis. In some embodiments, an effective amount of a composition having, as a therapeutic agent, a compound of the present approach, may be administered. In some embodiments, an effective amount of a composition having, as its therapeutic agent, an embodiment of a compound as described herein may be administered.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. The invention includes numerous alternatives, modifications, and equivalents as will become apparent from consideration of the following detailed description.

It will be understood that although the terms “first,” “second,” “third,” “a),” “b),” and “c),” etc. may be used herein to describe various elements of the invention, and the claims should not be limited by these terms. These terms are only used to distinguish one element of the invention from another. Thus, a first element discussed below could be termed an element aspect, and similarly, a third without departing from the teachings of the present invention. Thus, the terms “first,” “second,” “third,” “a),” “b),” and “c),” etc. are not intended to necessarily convey a sequence or other hierarchy to the associated elements but are used for identification purposes only. The sequence of operations (or steps) is not limited to the order presented in the claims.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also, as used herein, “and/or” refers to and encompasses any and all possible combinations of one or more of the associated listed items, as well as the lack of combinations when interpreted in the alternative (“or”).

The terms “decrease,” “lower,” “lessen,” and “reduce” generally refer to the ability of compositions according to the present approach to produce and/or cause a lesser physiological response (i.e., a measurable downstream effect), as compared to the response caused by either vehicle or a control molecule/composition, e.g., decreased tumor volume. A “decrease” or “reduced” response is typically a “statistically significant” response, and may include an decrease that is 1.1, 1.2, 1.5, 2, 3, 4, 5, 6, 7, 8, 9, 10, 15, 20, 30, or more times (e.g., 500, 1000 times) (including all integers and decimal points in between and above 1, e.g., 1.5, 1.6, 1.7. 1.8, etc.) the response produced by normal, untreated, or control-treated subject.

The phrase “treatment cycle” refers to a course of treatment, such as a dosing schedule that is repeated on a regular or pre-defined basis. A treatment cycle can comprise several days of treatment followed by several days of rest. For example only, an agent may be administered daily for two weeks, followed by two weeks of no treatment, over a 4-week treatment cycle. It should be appreciated that a treatment cycle may depend on a number of factors, such as the disease state, age, sex, and weight of the individual, as well as the particular agent(s) and/or methodologies, to elicit a desired response in the individual.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

The term “about,” as used herein when referring to a measurable value, such as, for example, an amount or concentration and the like, is meant to encompass variations of ±20%, ±10%, ±5%, ±1%, ±0.5%, or even ±0.1% of the specified amount. A range provided herein for a measurable value may include any other range and/or individual value therein.

Having thus described certain embodiments of the present invention, it is to be understood that the invention defined by the appended claims is not to be limited by particular details set forth in the above description as many apparent variations thereof are possible without departing from the spirit or scope thereof as hereinafter claimed.

1. A compound having the chemical structure: 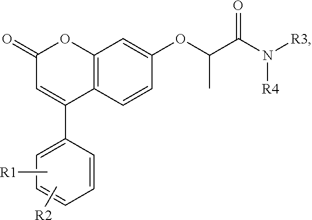
or a pharmaceutically acceptable salt thereof,
wherein:
R1 and R2 may be the same or different, and are selected from a halogen, CF₂H, —CF₃, —OCF₂H, —OCF₃, substituted or unsubstituted C5-C18 carboxyl, substituted or unsubstituted C5-C18 alkane, substituted or unsubstituted C5-C18 alkene, substituted or unsubstituted C5-C18 cyclic alkene, substituted or unsubstituted C5-C18 alkyne, substituted or unsubstituted C5-C18 ketone, substituted or unsubstituted C5-C18 aldehyde, substituted or unsubstituted C5-C18 ether, substituted or unsubstituted C5-C18 ester, substituted or unsubstituted C5-C18 amine, substituted or unsubstituted C5-C18 amide, substituted or unsubstituted C5-C18 alkyl-amide, monocyclic or polycyclic arene, heteroarene, phenol, or benzoic acid; and
R3 and R4 may be the same or different, and are selected from hydrogen, substituted or unsubstituted C2-C18-alkyl, substituted or unsubstituted C3-C8-cycloalkyl, substituted or unsubstituted pyridine, substituted or unsubstituted C2-C18 carboxyl, substituted or unsubstituted C2-C18 alkene, substituted or unsubstituted C2-C18 alkyne, substituted or unsubstituted C2-C18 ketone, substituted or unsubstituted C2-C18 aldehyde, substituted or unsubstituted C2-C18 ether, substituted or unsubstituted C2-C18 ester, substituted or unsubstituted C2-C18 amine, substituted or unsubstituted C2-C18 amide, substituted or unsubstituted C2-C18 alkyl-amide, substituted or unsubstituted phenol, or benzoic acid, or one of R3 and R4 is the group 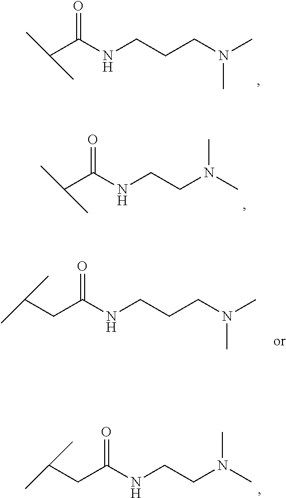
and the other is a H or a C2-C5 alkyl; or R3 and R4 form a substituted or unsubstituted C5 or C6 heterocycle; or R3 and R4 form the group 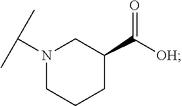
or NR3R4 is OH;
provided that at least one of R1 and R2 is a substituted or unsubstituted C5-C18 amide, or at least one of R3 and R4 is the group 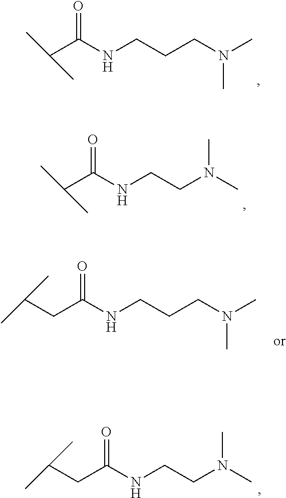
and the other is a H or a C2-C5 alkyl.

2. The compound of claim 1, having the structure: 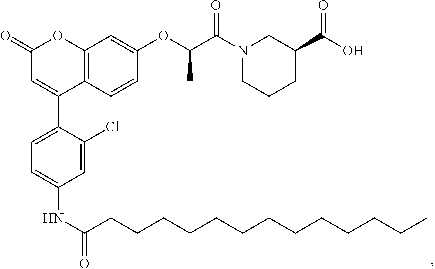 [SMILES: CCCCCCCCCCCCCC(=O)Nc1ccc(-c2cc(=O)oc3cc(O[C@H](C)C(=O)N4CCC[C@H](C(=O)O)C4)ccc23)c(Cl)c1]
or a pharmaceutically acceptable salt thereof.

3. The compound of claim 1, having the structure: 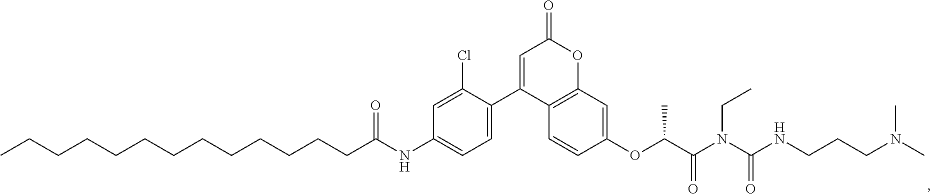 [SMILES: CCCCCCCCCCCCCC(=O)Nc1ccc(-c2cc(=O)oc3cc(O[C@H](C)C(=O)N(CC)C(=O)NCCCN(C)C)ccc23)c(Cl)c1]
or a pharmaceutically acceptable salt thereof.

4. The compound of claim 1, having the structure: 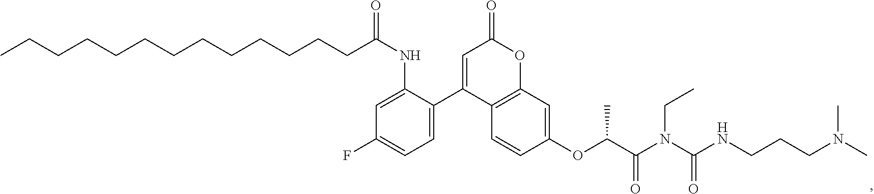 [SMILES: CCCCCCCCCCCCCC(=O)Nc1cc(F)ccc1-c1cc(=O)oc2cc(O[C@H](C)C(=O)N(CC)C(=O)NCCCN(C)C)ccc12]
or a pharmaceutically acceptable salt thereof.

5. The compound of claim 1, having the structure: 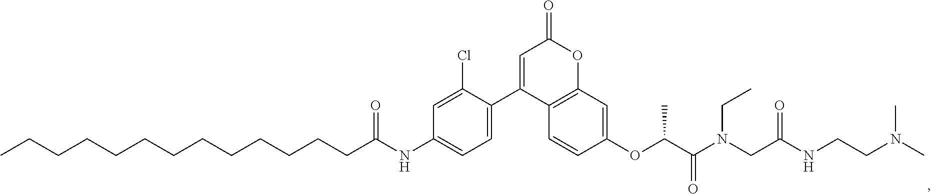 [SMILES: CCCCCCCCCCCCCC(=O)Nc1ccc(-c2cc(=O)oc3cc(O[C@H](C)C(=O)N(CC)CC(=O)NCCN(C)C)ccc23)c(Cl)c1]
or a pharmaceutically acceptable salt thereof.

6. The compound of claim 1, having the structure: 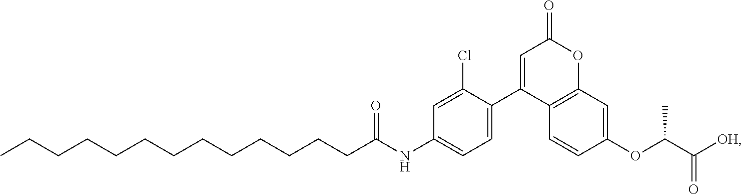 [SMILES: CCCCCCCCCCCCCC(=O)Nc1ccc(-c2cc(=O)oc3cc(O[C@H](C)C(=O)O)ccc23)c(Cl)c1]
or a pharmaceutically acceptable salt thereof.

7. The compound of claim 1, having the structure: 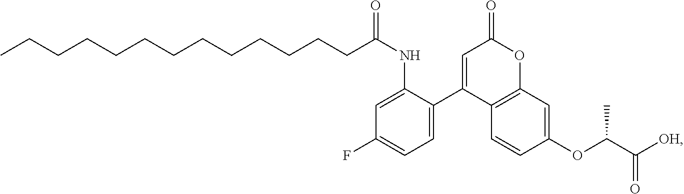 [SMILES: CCCCCCCCCCCCCC(=O)Nc1cc(F)ccc1-c1cc(=O)oc2cc(O[C@H](C)C(=O)O)ccc12]
or a pharmaceutically acceptable salt thereof.

8. The compound of claim 1, wherein one of R1 and R2 is a halogen.

9. The compound of claim 1, wherein one of R3 and R4 is the group 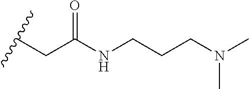 [SMILES: *CC(=O)NCCCN(C)C]
and the other is a C2-C5 alkyl.

10. The compound of claim 1, wherein one of R3 and R4 is the group 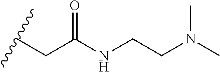 [SMILES: *CC(=O)NCCN(C)C]
and the other is a C2-C5 alkyl.

11. The compound of claim 1, wherein R3 and R4 form the group

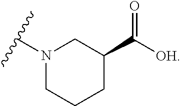 [SMILES: *N1CCC[C@H](C(=O)O)C1]

12. The compound of claim 1, having the structure: 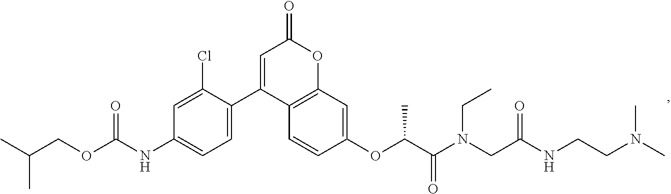 [SMILES: CCN(CC(=O)NCCN(C)C)C(=O)[C@@H](C)Oc1ccc2c(-c3ccc(NC(=O)OCC(C)C)cc3Cl)cc(=O)oc2c1]
or a pharmaceutically acceptable salt thereof.

13. The compound of claim 1, having the structure: 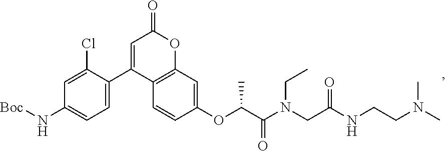 [SMILES: CCN(CC(=O)NCCN(C)C)C(=O)[C@@H](C)Oc1ccc2c(-c3ccc(NC(=O)OC(C)(C)C)cc3Cl)cc(=O)oc2c1]
or a pharmaceutically acceptable salt thereof.

14. The compound of claim 1, having the structure: 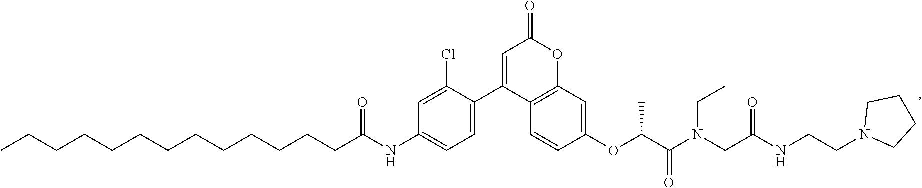 [SMILES: CCCCCCCCCCCCCC(=O)Nc1ccc(-c2cc(=O)oc3cc(O[C@H](C)C(=O)N(CC)CC(=O)NCCN4CCCC4)ccc23)c(Cl)c1]
or a pharmaceutically acceptable salt thereof.

15. The compound of claim 1, having the structure: 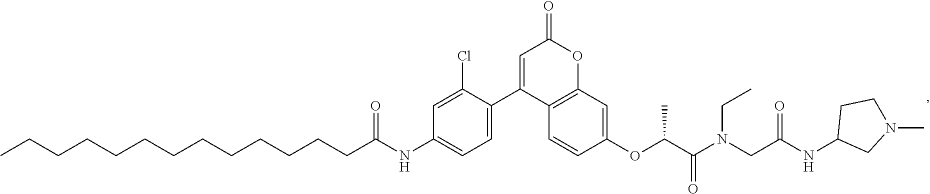 [SMILES: CCCCCCCCCCCCCC(=O)Nc1ccc(-c2cc(=O)oc3cc(O[C@H](C)C(=O)N(CC)CC(=O)NC4CCN(C)C4)ccc23)c(Cl)c1]
or a pharmaceutically acceptable salt thereof.

16. The compound of claim 1, having the structure: 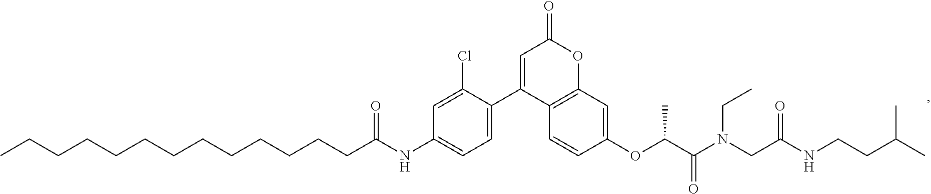 [SMILES: CCCCCCCCCCCCCC(=O)Nc1ccc(-c2cc(=O)oc3cc(O[C@H](C)C(=O)N(CC)CC(=O)NCCC(C)C)ccc23)c(Cl)c1]
or a pharmaceutically acceptable salt thereof.

17. A compound having the chemical structure: 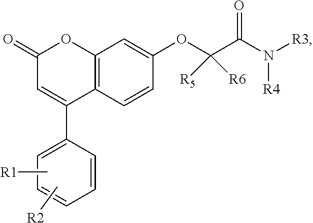
or a pharmaceutically acceptable salt thereof,
wherein:
R1 and R2 may be the same or different, and are selected from a halogen, CF₂H, —CF₃, —OCF₂H, —OCF₃, substituted or unsubstituted C5-C18 carboxyl, substituted or unsubstituted C5-C18 alkane, substituted or unsubstituted C5-C18 alkene, substituted or unsubstituted C5-C18 cyclic alkene, substituted or unsubstituted C5-C18 alkyne, substituted or unsubstituted C5-C18 ketone, substituted or unsubstituted C5-C18 aldehyde, substituted or unsubstituted C5-C18 ether, substituted or unsubstituted C5-C18 ester, substituted or unsubstituted C5-C18 amine, substituted or unsubstituted C5-C18 amide, substituted or unsubstituted C5-C18 alkyl-amide, monocyclic or polycyclic arene, heteroarene, phenol, or benzoic acid;
R5 and R6 may be the same or different, and are selected from hydrogen, substituted or unsubstituted C1-C10 alkyl, substituted or unsubstituted C3-C8-cycloalkyl, substituted or unsubstituted pyridine, substituted or unsubstituted C2-C10 carboxyl, substituted or unsubstituted C2-C10 alkene, substituted or unsubstituted C2-C10 alkyne, substituted or unsubstituted C2-C10 ketone, substituted or unsubstituted C2-C10 aldehyde, substituted or unsubstituted C2-C10 ether, substituted or unsubstituted C2-C10 ester, substituted or unsubstituted C2-C10 amine, substituted or unsubstituted C2-C10 amide, substituted or unsubstituted C2-C10 alkyl-amide, substituted or unsubstituted phenol, or benzoic acid; and
R3 and R4 may be the same or different, and are selected from hydrogen, substituted or unsubstituted C2-C18-alkyl, substituted or unsubstituted C3-C8-cycloalkyl, substituted or unsubstituted pyridine, substituted or unsubstituted C2-C18 carboxyl, substituted or unsubstituted C2-C18 alkene, substituted or unsubstituted C2-C18 alkyne, substituted or unsubstituted C2-C18 ketone, substituted or unsubstituted C2-C18 aldehyde, substituted or unsubstituted C2-C18 ether, substituted or unsubstituted C2-C18 ester, substituted or unsubstituted C2-C18 amine, substituted or unsubstituted C2-C18 amide, substituted or unsubstituted C2-C18 alkyl-amide, substituted or unsubstituted phenol, or benzoic acid, or one of R3 and R4 is the group 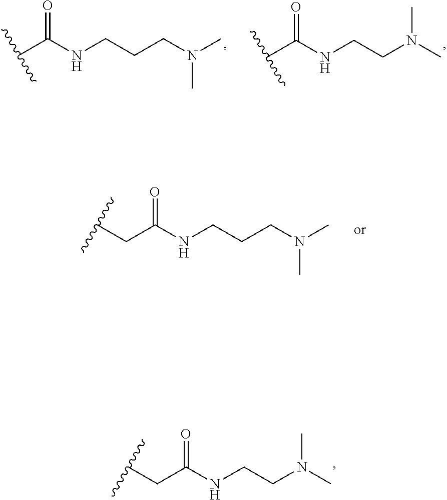
and the other is a H or a C2-C5 alkyl; or R3 and R4 form a substituted or unsubstituted C5 or C6 heterocycle; or R3 and R4 form the group 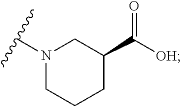 [SMILES: *N1CCC[C@H](C(=O)O)C1]
or NR3R4 is OH;
provided that at least one of R1 and R2 is a substituted or unsubstituted C5-C18 amide, or at least one of R3 and R4 is the group, 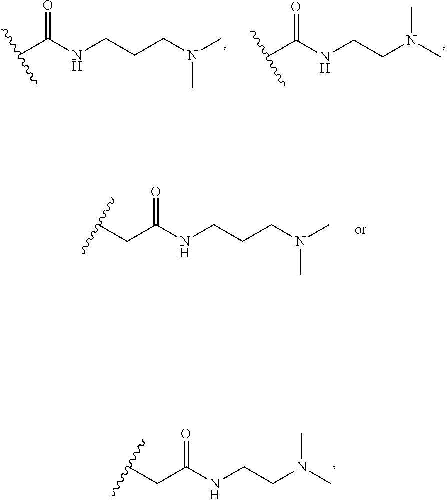
and the other is a H or a C2-C5 alkyl.

18. The compound of claim 17, having the structure: 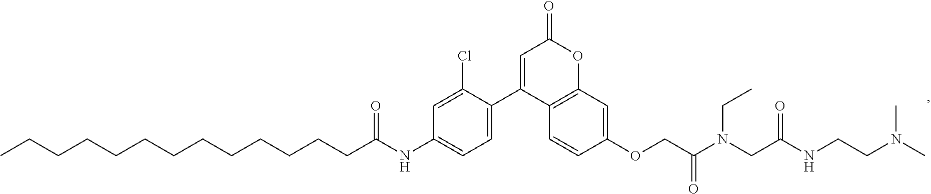 [SMILES: CCCCCCCCCCCCCC(=O)Nc1ccc(-c2cc(=O)oc3cc(OCC(=O)N(CC)CC(=O)NCCN(C)C)ccc23)c(Cl)c1]
or a pharmaceutically acceptable salt thereof.

19. The compound of claim 17, wherein R5 and R6 are selected from the group consisting of:

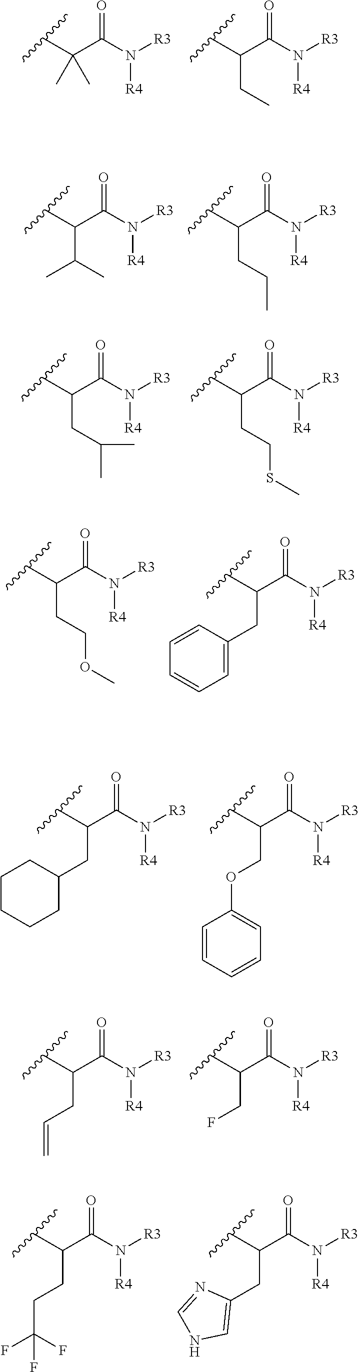

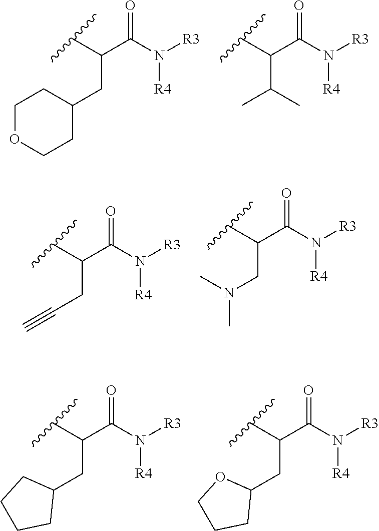

20. A pharmaceutical composition comprising a pharmaceutically effective amount of the compound of claim 1, and a pharmaceutically acceptable carrier.

21. A method for treating or preventing tumor recurrence and/or metastasis, the method comprising administering to a patient at risk of tumor recurrence and/or metastasis a pharmaceutically effective amount of a pharmaceutical composition comprising the compound of claim 1.

22. The method of claim 16, wherein the administering is performed at least one of prior to a cancer treatment, with a cancer treatment, and following a cancer treatment.

23. A method for inhibiting the propagation of cancer stem cells in a patient, the method comprising administering to the patient a pharmaceutically effective amount of a pharmaceutical composition comprising the compound of claim 1.

24. The method of claim 18, wherein the administering is performed at least one of prior to a cancer treatment, with a cancer treatment, and following a cancer treatment.

25. A method for treating cancer, the method comprising: administering to a person having cancer a pharmaceutically effective amount of a pharmaceutical composition comprising the compound of claim 1.

26. The method of claim 18, wherein the administering is performed at least one of prior to a cancer treatment, with a cancer treatment, and following a cancer treatment.
